(19) United States
(12) Patent Application Publication
(10) Pub. No.: US 2023/0233326 A1
(43) Pub. Date: Jul. 27, 2023

(54) VALVE LEAFLET CINCHING

(71) Applicant: EDWARDS LIFESCIENCES CORPORATION, Irvine, CA (US)

(72) Inventors: Brian S. Conklin, Orange, CA (US); Maria L. Saravia, Irvine, CA (US); Harvey H. Chen, Irvine, CA (US); Cindy Woo, Irvine, CA (US)

(21) Appl. No.: 18/190,025

(22) Filed: Mar. 24, 2023

Related U.S. Application Data
(63) Continuation of application No. PCT/US2021/051880, filed on Sep. 24, 2021.
(60) Provisional application No. 63/172,477, filed on Apr. 8, 2021.
(60) Provisional application No. 63/083,601, filed on Sep. 25, 2020.

(51) Int. Cl. A61F 2/24; A61B 17/04

(52) U.S. Cl. CPC A61F 2/2466; A61F 2/2457; A61B 17/0401; A61F 2220/0075; A61F 2220/0008; A61B 2017/0406; A61B 2017/0409; A61B 2017/0464; A61B 2017/00243

(57) ABSTRACT

A method comprises inserting a needle into a ventricle of a heart. The needle is configured to deliver a first anchoring element, a second anchoring element, and a tethering suture to the ventricle. The tethering suture is tethered between the first anchoring element and the second anchoring element. The method further comprises penetrating a first leaflet of a heart valve with the needle, penetrating a second leaflet of the heart valve with the needle, deploying the first anchoring element at a distal side of the second leaflet, retracting the needle from the first leaflet and the second leaflet, deploying the second anchoring element at a proximal side of the first leaflet, cinching the tethering suture to cause a desired amount of valve coaptation, and locking the tethering suture.

### RELATED APPLICATIONS

This application is a continuation of International Patent Application No. PCT/US2021/051880, filed Sep. 24, 2021, which claims the benefit of U.S. Application No. 63/083,601, filed on Sep. 25, 2020, and of U.S. Application No. 63/172,477, filed on Apr. 8, 2021, the disclosures all of which are incorporated by reference in their entireties.

### BACKGROUND

The present disclosure generally relates to the field of improving heart performance.

Heart failure with reduced ejection fraction (HFrEF), also known as systolic heart failure, is characterized by an inability of the heart to contract adequately, resulting in less oxygen-rich blood being expelled into the body. Functional mitral valve regurgitation (FMR) is a disease that occurs when the left ventricle of the heart is distorted or dilated, displacing the papillary muscles that support the two valve leaflets. When the valve leaflets can no longer come together to close the annulus, blood may flow back into the atrium.

### SUMMARY

For purposes of summarizing the disclosure, certain aspects, advantages and novel features have been described herein. It is to be understood that not necessarily all such advantages may be achieved in accordance with any particular instance. Thus, the disclosed instances may be carried out in a manner that achieves or optimizes one advantage or group of advantages as taught herein without necessarily achieving other advantages as may be taught or suggested herein.

Some implementations of the present disclosure relate to a method comprising inserting a needle into a ventricle of a heart. The needle is configured to deliver a first anchoring element, a second anchoring element, and a tethering suture to the ventricle. The tethering suture is tethered between the first anchoring element and the second anchoring element. The method further comprises penetrating a first leaflet of a heart valve with the needle, penetrating a second leaflet of the heart valve with the needle, deploying the first anchoring element at a distal side of the second leaflet, retracting the needle from the first leaflet and the second leaflet, deploying the second anchoring element at a proximal side of the first leaflet, cinching the tethering suture to cause a desired amount of valve coaptation, and locking the tethering suture.

In some instances, the needle may be inserted into the ventricle via a septum. The needle may be inserted into the ventricle via a posterior wall of the heart.

The first anchoring element may comprise a first sutureform wrapped at least partially around the needle. In some instances, the tethering suture extends from the first sutureform.

In some instances, the second anchoring element comprises a second sutureform. The tethering suture may be configured to pass at least partially through a lumen formed by coils of the second sutureform.

The method may further comprise extending a pusher along an outer surface of the needle to press the first anchoring element and the second anchoring element off of the needle. In some instances, the first anchoring element is configured to form a knot when removed from the needle.

In some instances, the needle comprises an internal lumen and an aperture to the internal lumen. The needle may be configured to deploy the first anchoring element via the internal lumen and the aperture.

The first anchoring element may be configured to form a non-continuous circular shape when removed from the needle.

In some instances, the first anchoring element comprises a base portion, a shaping device configured to fit within the base portion, and two or more appendages extending from the base portion. The tethering suture may be configured to pass through each of the two or more appendages to pull the two or more appendages radially inward towards a central area of the first anchoring element.

The shaping device may be at least partially composed of a shape-memory material.

In accordance with some implementations of the present disclosure, a system comprises a first anchoring element, a second anchoring element, a tethering suture tethered between the first anchoring element and the second anchoring element, and a needle configured to penetrate a first leaflet of a heart valve within a ventricle of a heart, penetrate a second leaflet of the heart valve, deliver the first anchoring element at a distal side of the second leaflet, and deliver the second anchoring element at a proximal side of the first leaflet.

The first anchoring element may comprise a first sutureform wrapped at least partially around the needle. In some instances, the system further comprises a pusher configured to extend along an outer surface of the needle to press the first anchoring element and the second anchoring element off of the needle.

In some instances, the needle comprises an internal lumen and an aperture to the internal lumen and the needle is further configured to deliver the first anchoring element via the internal lumen and the aperture. The first anchoring element may be configured to form a non-continuous circular shape when removed from the needle.

The first anchoring element may comprise a base portion, a shaping device configured to fit within the base portion, and two or more appendages extending from the base portion. In some instances, the tethering suture is configured to pass through each of the two or more appendages to pull the two or more appendages radially inward towards a central area of the first anchoring element.

Some implementations of the present disclosure relate to a method comprising inserting a needle carrying a first suture anchoring element, a second suture anchoring element, and a pusher to a proximal side of a valve of a heart. The first suture anchoring element and the second suture anchoring element are at least partially situated within the pusher. The method further comprises retracting the pusher to expose the first suture anchoring element and extending the pusher to press the first suture anchoring element through a leaflet of the valve.

In some instances, the method further comprises retracting the pusher to expose the second suture anchoring element and extending the pusher to press the second suture anchoring element through the leaflet of the valve. The method may further comprise retracting the pusher to expose the second suture anchoring element and extending the pusher to press the second suture anchoring element off of the needle.

The first suture anchoring element and the second suture anchoring element may comprise a single suture. In some instances, the first suture anchoring element and the second suture anchoring element comprise separate sutures.

In accordance with some implementations of the present disclosure, a device comprises a main body portion having a first diameter. The main body portion is configured to receive a suture anchoring element wrapped around a delivery device. The device further comprises an elastic tip having, at a distal end portion of the elastic tip, a second diameter that is less than the first diameter and less than a diameter of the suture anchoring element wrapped around the delivery device. The elastic tip is configured to at least partially expand to allow the suture anchoring element to exit the main body portion and the elastic tip and at least partially compress to the second diameter to press against the suture anchoring element.

The elastic tip may comprise one or more slits extending from the distal end portion of the elastic tip to the main body portion. In some instances, the elastic tip is configured to naturally assume a tapered shape in which the elastic tip gradually increases in diameter from the distal end portion to the main body portion.

Some implementations of the present disclosure relate to a method comprising inserting a needle into a ventricle of a heart. The needle is configured to deliver a suture forming a first anchoring element, a second anchoring element, and a tethering suture to the ventricle. The tethering suture is tethered between the first anchoring element and the second anchoring element. The method further comprises penetrating a first leaflet of a heart valve with the needle, penetrating a second leaflet of the heart valve with the needle, deploying the first anchoring element at a distal side of the second leaflet, retracting the needle from the first leaflet and the second leaflet, and deploying the second anchoring element at a proximal side of the first leaflet.

In some instances, the method further comprises cinching the tethering suture to cause a desired amount of valve coaptation and locking the tethering suture. The suture may be further configured to form a first knot between the first anchoring element and the second anchoring element.

The suture may be further configured to form a second knot between the first anchoring element and the second anchoring element. In some instances, the tethering suture is configured to pass under one or more coils of the first anchoring element.

In some instances, the first anchoring element is configured to form a knot when removed from the needle. The second anchoring element may be configured to form a knot when removed from the needle.

The method may further comprise retracting a pusher to expose the first anchoring element and extending the pusher to deploy the first anchoring element.

In accordance with some implementations of the present disclosure, a method comprises inserting a needle carrying a first suture anchoring element, a second suture anchoring element, and a pusher to a proximal side of a valve of a heart. The first suture anchoring element and the second suture anchoring element are at least partially situated within the pusher. The method further comprises retracting the pusher to expose the first suture anchoring element and extending the pusher to press the first suture anchoring element through a leaflet of the valve.

The method may further comprise retracting the pusher to expose the second suture anchoring element and extending the pusher to press the second suture anchoring element through the leaflet of the valve. In some instances, the method further comprises retracting the pusher to expose the second suture anchoring element and extending the pusher to press the second suture anchoring element off of the needle.

In some instances, the first suture anchoring element is configured to form a knot when removed from the needle. The first suture anchoring element and the second suture anchoring element may comprise a single suture.

The first suture anchoring element and the second suture anchoring element may comprise separate sutures.

Some implementations of the present disclosure relate to a device comprising a main body portion having a first diameter. The main body portion is configured to receive a suture anchoring element wrapped around a delivery device. The device further comprises an elastic tip having, at a distal end portion of the elastic tip, a second diameter that is less than the first diameter and less than a diameter of the suture anchoring element wrapped around the delivery device. The elastic tip is configured to at least partially expand to allow the suture anchoring element to exit the main body portion and the elastic tip and at least partially compress to the second diameter to press against the suture anchoring element.

In some instances, the main body portion is configured to receive two or more suture anchoring elements. The elastic tip may comprise one or more slits extending from the distal end portion of the elastic tip to the main body portion.

The distal end portion of the elastic tip may be flat to allow the elastic tip to push the suture anchoring element off of the delivery device. In some instance, the elastic tip may be configured to naturally assume a tapered shape in which the elastic tip gradually increases in diameter from the distal end portion to the main body portion.

In some instances, the device further comprises an elastic sleeve configured to at least partially surround the elastic tip and assist in compressing the elastic tip.

In accordance with some implementations of the present disclosure, a heart valve anchoring system comprises a needle comprising a pointed tip at a distal end portion of the needle. The pointed tip is configured to puncture a leaflet a of a heart valve. The anchoring system further comprises a first suture anchor wrapped around an exterior surface of the needle near the distal end portion of the needle and a second suture anchor wrapped around the exterior surface of the needle. The first suture anchor is situated between the second suture anchor and the distal end portion of the needle. The anchoring system further comprises a pusher configured to press against a proximal side of the first suture anchor to push the first suture anchor off of the needle, retract over the second suture anchor, and press against a proximal side of the second suture anchor to push the second suture anchor off of the needle.

The first suture anchor may comprise a first suture having a first end and a second end, and wherein the first suture is configured to form multiple coils along the needle between a distal side of the first suture anchor and the proximal side of the first suture anchor. In some instances, the first end of the first suture is configured to pass under one or two coils of the first suture anchor and over all other coils of the first suture anchor.

In some instances, the first end of the first suture and the second end of the first suture are configured to form a knot at the proximal side of the first suture anchor.

The first suture may be configured to form the second suture anchor. In some instances, the first suture may be configured to form multiple coils along the needle between a distal side of the second suture anchor and the proximal side of the second suture anchor.

In some instances, the first end of the first suture and the second end of the first suture are configured to form a first knot at the distal side of the second suture anchor. The first end of the first suture may be configured to pass under one or two coils of the second suture anchor and over all other coils of the second suture anchor.

The first end of the first suture and the second end of the first suture may be configured to form a second knot at the proximal side of the second suture anchor.

### BRIEF DESCRIPTION OF THE DRAWINGS

Various instances are depicted in the accompanying drawings for illustrative purposes, and should in no way be interpreted as limiting the scope of the inventions. In addition, various features of different disclosed instances can be combined to form additional instances, which are part of this disclosure. Throughout the drawings, reference numbers may be reused to indicate correspondence between reference elements. However, it should be understood that the use of similar reference numbers in connection with multiple drawings does not necessarily imply similarity between respective instances associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some instances or configurations.

FIG. 1 provides a cross-sectional view of a human heart.

FIG. 2 provides a cross-sectional view of the left ventricle and left atrium of an example heart.

FIG. 3 provides a cross-sectional view of a heart experiencing mitral regurgitation.

FIGS. 4A-1, 4A-2, 4B-1, 4B-2, and 4C provide perspective views of a coaptation device (including one or more anchoring elements and sutures) implanted at a mitral valve of a heart in accordance with one or more examples.

FIG. 5 shows a close-up view of a shaft of a tissue anchor delivery device inserted into a ventricle and approximated to a target valve leaflet in connection with a valve-repair procedure in accordance with one or more instances of the present disclosure.

FIG. 6 shows a cutaway view of multiple deployed leaflet anchors in accordance with one or more instances of the present disclosure.

FIG. 7 illustrates a coaptation device comprising a first anchoring element and a second anchoring element in accordance with one or more examples.

FIG. 8 illustrates another coaptation device comprising a first anchoring element and a second anchoring element in accordance with one or more examples.

FIG. 9 illustrates another coaptation device comprising a first anchoring element and a second anchoring element in accordance with one or more examples.

FIG. 10 illustrates another coaptation device comprising a first anchoring element and a second anchoring element in accordance with one or more examples.

FIG. 11 illustrates another coaptation device comprising a first anchoring element and a second anchoring element in accordance with one or more examples.

FIG. 12 illustrates another coaptation device comprising a first anchoring element and a second anchoring element in accordance with one or more examples.

FIGS. 13A-13C illustrate an example anchoring element which may be delivered to one or more valve leaflets in accordance with one or more examples.

FIG. 14 illustrates an example coaptation device comprising multiple anchoring elements in accordance with some examples.

FIG. 15 illustrates another example coaptation device comprising multiple anchoring elements in accordance with some examples.

FIG. 16 illustrates another example coaptation device comprising multiple anchoring elements locked in place by a tethering suture in accordance with some examples.

FIG. 17 illustrates an example delivery system for delivering one or more anchoring elements to a valve leaflet in accordance with some examples.

FIGS. 18A-18C illustrate another delivery system for delivering one or more anchoring elements to a valve leaflet in accordance with one or more examples.

FIG. 19 (19-1, 19-2, and 19-3) provides a flow diagram representing a process for repairing a valve of the heart according to one or more instances disclosed herein.

FIG. 20 (20-1, 20-2, and 20-3) shows examples of various stages of the process for repairing a heart valve shown in FIG. 19.

FIG. 21 shows a coaptation device comprising a first anchoring element and a second anchoring element in accordance with one or more examples.

FIG. 22 illustrates a coaptation device comprising two anchoring elements forming bulky knot anchors in accordance with one or more examples.

FIG. 23 (23-1 and 23-2) provides a flow diagram representing a process for wrapping a suture onto a delivery device according to one or more instances disclosed herein.

FIG. 24 (24-1 and 24-2) shows examples of various stages of the process for wrapping the suture onto the delivery device shown in FIG. 23 in accordance with one or more examples.

FIG. 25 illustrates an example delivery system for delivering one or more anchoring elements to a valve leaflet in accordance with some examples.

FIG. 26 illustrates a pusher which may be configured to deploy one or more anchoring elements at one or more valve leaflets in accordance with one or more instances.

FIG. 27 (27-1, 27-2, and 27-3) provides a flow diagram representing a process for delivery of one or more anchoring elements to a valve of a heart according to one or more instances disclosed herein.

FIG. 28 (28-1, 28-2, and 28-3) shows examples of various stages of the process for delivery of one or more anchoring elements to a valve of a heart shown in FIG. 27 according to one or more instances disclosed herein.

FIG. 29 (29-1 and 29-2) provides a flow diagram representing a process for delivery of one or more anchoring elements to a valve of a heart according to one or more instances disclosed herein.

FIG. 30 (30-1 and 30-2) shows examples of various stages of the process for delivery of one or more anchoring elements to a valve of a heart shown in FIG. 29 according to one or more instances disclosed herein.

FIG. 31 illustrates an overhead view of an upper/distal side of a valve having two anchoring elements with a bulky knot form delivered to the upper/distal side of the valve according to one or more instances disclosed herein.

FIG. 32 illustrates an overhead view of an upper/distal side of a valve having six anchoring elements with a bulky knot form delivered to the upper/distal side of the valve according to one or more instances disclosed herein.

### DETAILED DESCRIPTION

The headings provided herein are for convenience only and do not necessarily affect the scope or meaning of the claimed invention.

Although certain preferred instances and examples are disclosed below, inventive subject matter extends beyond the specifically disclosed instances to other alternative instances and/or uses and to modifications and equivalents thereof. Thus, the scope of the claims that may arise herefrom is not limited by any of the particular instances described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain instances; however, the order of description should not be construed to imply that these operations are order dependent. Additionally, the structures, systems, and/or devices described herein may be embodied as integrated components or as separate components. For purposes of comparing various instances, certain aspects and advantages of these instances are described. Not necessarily all such aspects or advantages are achieved by any particular instance. Thus, for example, various instances may be carried out in a manner that achieves or optimizes one advantage or group of advantages as taught herein without necessarily achieving other aspects or advantages as may also be taught or suggested herein.

The following description refers to the accompanying drawings, which illustrate specific instances. Other instances having different structures and operation do not depart from the scope of the disclosure.

### Overview

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary artery, aorta, etc.).

Disclosed herein are suture-tensioning devices, which can have a generally elongate shape or form, at least in part. The devices can be used to facilitate or promote relatively precise tensioning of sutures (e.g., suture portions) anchored to biological tissue such as to one or more mitral valve leaflets. Although aspects of the present disclosure are presented in the context of sutures associated with tissue anchoring elements (e.g., valve leaflet repair tissue anchoring elements), it should be understood that tensioning device in accordance with the present disclosure may be used for tensioning any type of suture or other elongate form/member.

The term “suture” is used herein according to its plain and ordinary meaning and may refer to any elongate cord strip, strand, line, rope, wire, filament, tie, string, ribbon, strap, or portion thereof, or other type/form of material used in medical procedures (e.g., ePTFE suture, for example, GORE-TEX® ePTFE suture (W.L. Gore, Newark, Del.); polyester suture, for example, ETHIBOND® polyester suture (Ethicon); polypropylene suture; ultra-high-molecular-weight polyethylene (UHMWPE) suture, for example, FORCE FIBER® suture (Teleflex, Gurnee, Ill.); etc.). Furthermore, examples of the present disclosure may be implemented in connection with non-surgical and/or non-biological suture/line tensioning. With respect to the present disclosure, one having ordinary skill in the art will understand that a wire or other similar material may be used in place of a suture. Furthermore, in some contexts herein, the terms “cord” and “suture” may be used substantially interchangeably. In addition, use of the singular form of any of the suture-related terms listed above, including the terms “suture” and “cord,” may be used to refer to a single suture/cord, or to a portion thereof. For example, where a suture knot or anchoring element is deployed on a distal side of a tissue portion, and where two suture portions extend from the knot/anchoring element on a proximal side of the tissue, either of the suture portions may be referred to as a “suture” or a “cord,” regardless of whether both portions are part of a unitary suture or cord. Suture tensioning devices in accordance with aspects of the present disclosure may be utilized in methods for tensioning surgical sutures deployed in a ventricle and/or atrium of a heart. Such sutures and/or associated anchoring elements may be introduced to the target implantation site using a minimally invasive incision, as described in greater detail below.

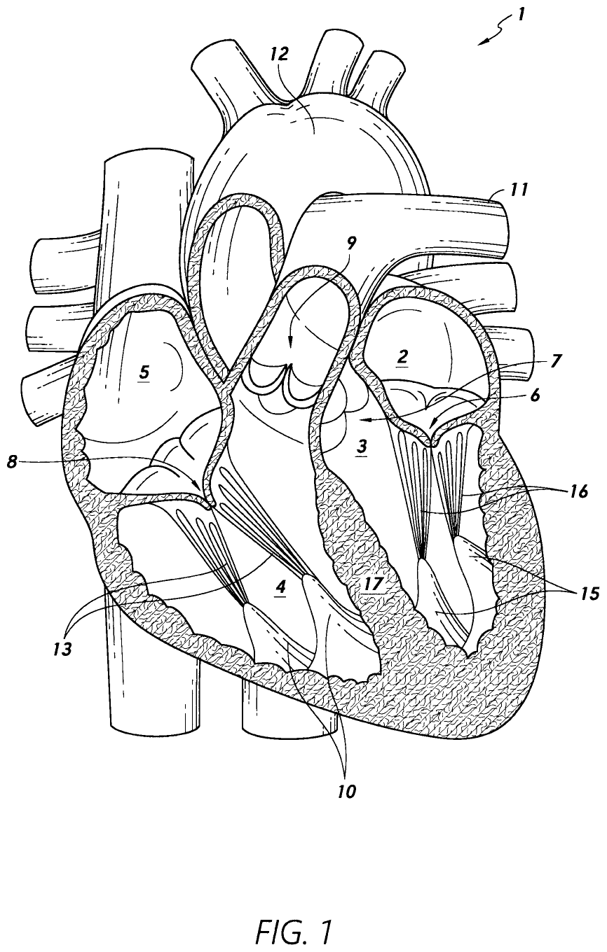

FIG. 1 illustrates an example representation of a heart 1 having various features relevant to certain instances of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (e.g., mitral and tricuspid) heart valves may further comprise a collection of chordae tendineae and papillary muscles for securing the leaflets of the respective valves to promote and/or facilitate proper repairing of the valve leaflets and prevent prolapse thereof. The papillary muscles, for example, may generally comprise finger-like projections from the ventricle wall. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles 10 by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall, and attach to the anterior, posterior and septal leaflets of the tricuspid valve, respectively, via the chordae tendineae 13. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. The various chords of the chordae tendineae may have different thicknesses, wherein relatively thinner chords are attached to the free leaflet margin, while relatively thicker chords (e.g., strut chords) are attached farther away from the free margin.

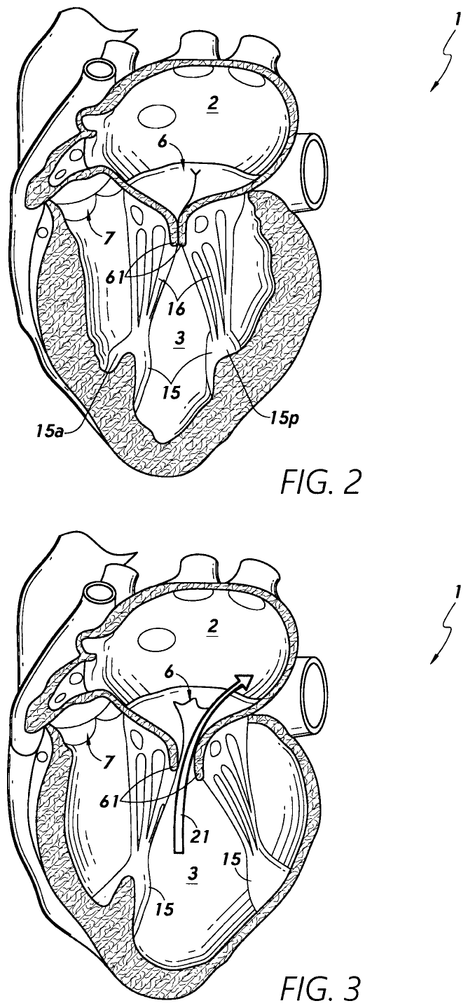

FIG. 2 provides a cross-sectional view of the left ventricle 3 and left atrium 2 of an example heart 1. The diagram of FIG. 2 shows the mitral valve 6, wherein the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2 and various other figures provided herewith and described herein in the context of certain instances of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve as shown in FIG. 2, the valve leaflets 61 may extend inward from the valve annulus and come together in the flow orifice to permit flow in the outflow direction (e.g., the downward direction in FIG. 2) and prevent backflow or regurgitation toward the inflow direction (e.g., the upward direction in FIG. 2). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets in a closed position. The papillary muscles 15 may include a first papillary muscle 15a (e.g., an anterolateral papillary muscle, which may be primarily tethered to the anterior leaflet, for example) and a second papillary muscle 15p (e.g., the posteromedial papillary muscle, which may be primarily tethered to the posterior leaflet, for example). Each of the first papillary muscle 15a and second papillary muscle 15p may provide chordae tendineae 16 to each valve leaflet (e.g., the anterior and posterior leaflets). With respect to the state of the heart 1 shown in FIG. 2, the proper repairing of the valve leaflets, which may be due in part to proper position of the papillary muscles 15, may advantageously result in mitral valve operation substantially free of leakage.

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3, which illustrates a cross-sectional view of a heart 1 experiencing mitral regurgitation flow 21, dilation of the left ventricle may cause changes in the position of the papillary muscles 15 that allow flow 21 back from the ventricle 3 to the atrium 2. Dilation of the left ventricle can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

With further reference to FIG. 3, the heart 1 is shown in a state where functional mitral valve regurgitation (FMR) is present. FMR may be considered a disease of the left ventricle 3, rather than of the mitral valve 6. For example, mitral valve regurgitation may occur when the left ventricle 3 of the heart 1 is distorted or dilated, displacing the papillary muscles 15 that support the two valve leaflets 61. The valve leaflets 61 therefore may no longer come together sufficiently to close the annulus and prevent blood flow back into the atrium 2. If left untreated, the FMR experienced in the state shown in FIG. 3 may overload the heart 1 and can possibly lead to or accelerate heart failure. Solutions presented herein provide devices and methods for moving the papillary muscles 15 closer to their previous position, which may advantageously reduce the occurrence of mitral regurgitation.

As shown in FIG. 3, the leaflets 61 of the mitral valve (or tricuspid valve) are not in a state of repairing, resulting in an opening between the mitral valve leaflets 61 during the systolic phase of the cardiac cycle, which allows the leakage flow 21 of fluid back up into the atrium 2. The papillary muscles 15 may be displaced due to dilation of the left ventricle 3, or due to one or more other conditions, as described above, which may contribute to the failure of the valve 6 to close properly. The failure of the valve leaflets 61 to coapt properly may result in unwanted flow in the outflow direction (e.g., the upward direction in FIG. 3) and/or unwanted backflow or regurgitation toward the inflow direction (e.g., the downward direction in FIG. 2).

Some methods for restoring function to a regurgitant valve (e.g., mitral valve) include open-heart, on-pump repair, and/or minimally invasive procedures. In some cases, valve leaflets can be reshaped as needed and/or support structures (e.g., annuloplasty bands) can be added to reduce the size of the annulus. Some methods involve edge-to-edge repair techniques (e.g., an Alfieri stitch) to create a permanent area of coaptation between the two leaflets and/or form a double orifice. Some open and/or on-pump repair procedures can require arresting the patient's heart and maintaining life support with a heart lung machine. Moreover, in some cases, surgeons may perform such procedures rarely (e.g., a few times per year) and may lack the necessary experience to become proficient in valve repair, which may lead to poor outcomes.

In some methods (e.g., “water tests”), surgeons may inject water with a syringe into a ventricle and observe whether the water leaks back through a repaired valve. Only after closing and restarting the patient's heart can the surgeon determine the efficacy of the repair, which can be poor even in spite of a successful water test. Some procedures involve joining the valve leaflets together at a point on the coaptation edges by clipping the leaflets together. This results in a double orifice and/or potentially compromised hemodynamics. In some cases, clips used for such procedures may be expensive and/or bulky and/or several implants may be required.

Some examples described herein involve devices that can be used to perform an edge-to-edge valve annuloplasty (e.g., mitral annuloplasty) on a beating heart through a percutaneous and/or beating heart surgical approach. In some examples, a valve repair procedure may advantageously require only a single implant (e.g., two anchoring elements tethered by a tethering element).

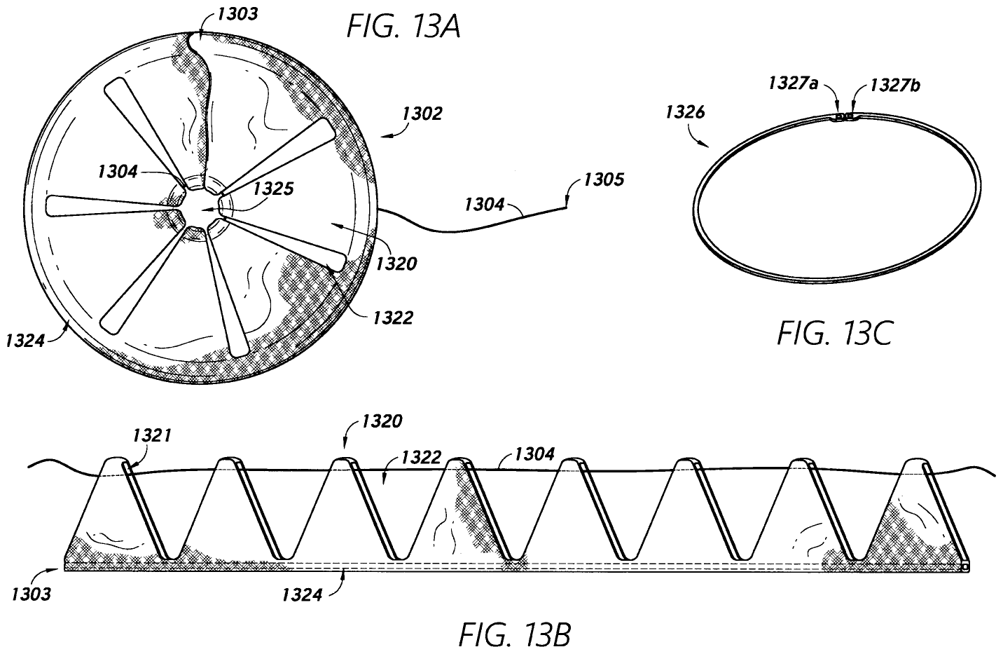

In some instances, a device may utilize multiple anchoring elements (e.g., sutureforms) wrapped in series around a delivery device (e.g., an at least partially hollow needle). The delivery device and/or one or more anchoring elements may be passed through one or more valve leaflets to situate, for example, a first anchoring element at a distal side of a distal leaflet and/or a second anchoring element at a proximal side of a proximal leaflet. In some instances, one or more anchoring elements may additionally or alternatively be delivered via a hollow interior (e.g., an interior lumen) of the delivery device. The one or more anchoring elements may comprise one or more disc anchors (e.g., cloth-covered disc devices; see, e.g., FIGS. 13A-13C). In some instances, one or more anchoring elements may comprise one or more shape-memory components, for example a Nitinol core.

Some instances disclosed herein provide solutions for treating FMR and/or heart failure with reduced ejection fraction (HFrEF) without the need for surgical procedures or destroying cardiac tissue. In particular, passive techniques to improve valve performance are disclosed for improving cardiac function. Furthermore, various instances disclosed herein provide for the treatment of FMR and/or HFrEF that can be executed on a beating heart, thereby allowing for the ability to assess the efficacy of the treatment and potentially implement modification thereto without the need for bypass support.

Some instances involve performing an annuloplasty and/or otherwise placating multiple tissue surfaces (e.g., valve leaflets) to restore valve function and/or improve ejection fraction. Valve repair (e.g., at least partially closing a leaking valve) can potentially treat FMR and/or HFrEF. Some instances described herein involve repairing valves (e.g., the mitral valve) by inserting one or more sutures, which may include bands, cords, strings, tubes, or other lengths of material (referred to herein collectively as “sutures,” “cords,” “tethering sutures,” “tethering members,” or “means for tethering”) into a ventricle and anchoring the suture(s) to multiple leaflets of the valve. By tightening the suture(s), the leaflets may be brought together at least at one point.

The suture(s) may be anchored through use of anchoring elements that may directly contact and/or anchor the suture(s) to one or more leaflets and/or other areas of tissue. In some instances, the suture(s) may be tightened to reduce a distance between anchoring elements at the multiple leaflets.

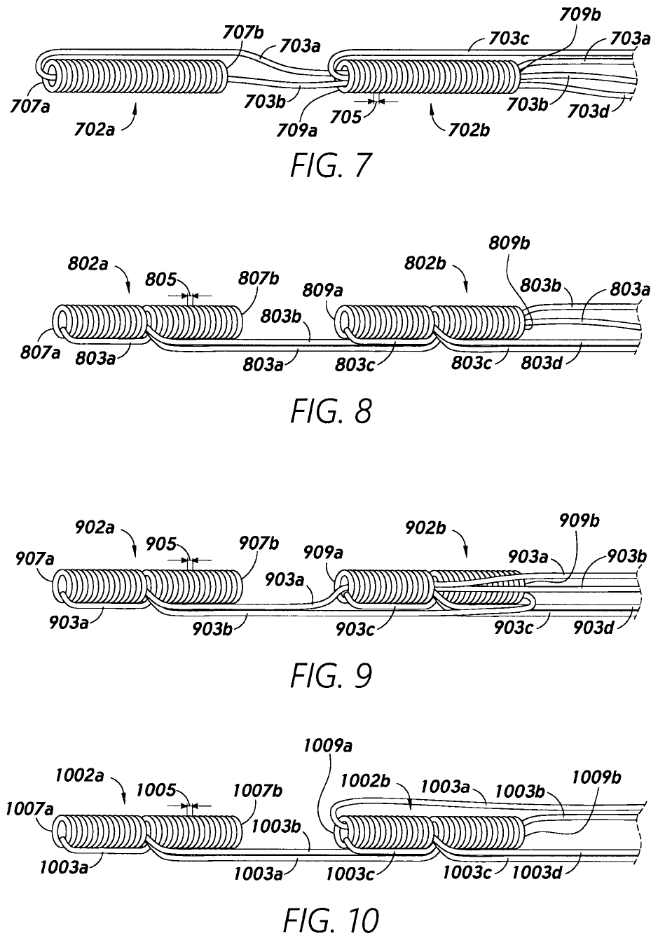

In some instances, anchoring elements may be at least partially composed of sutures (e.g., sutureforms and/or “bulky knots”; see, e.g., FIGS. 7-12) and/or various fabrics and/or other materials. Anchoring elements may be at least partially flexible and/or elastic to allow the anchoring elements to be compressed into a delivery device (e.g., a needle) and/or expanded when delivered to a target valve. When a suture is cinched, pressure may be applied to each of the anchoring elements in order to reduce strain at any one individual anchor. Multiple types of anchoring elements may be used to anchor a suture to a valve leaflet. For example, a penetration element (e.g., a needle) may be used to deliver a first type of anchoring element to a first (e.g., distal) leaflet and/or to a second (e.g., proximal) leaflet.

The term “needle” is used herein according to its broad and ordinary meaning and may refer to an anchoring device/element having a pointed end and configured to penetrate a tissue wall with the pointed end. In some instances, the needle may have an at least partially conical shape. The needle may be solid or at least partially hollow.

### Valve Repair Devices

FIGS. 4A-1, 4A-2, 4B-1, 4B-2, and 4C provide perspective views of a coaptation device 400 including one or more anchoring elements 402 and sutures 404 implanted at a mitral valve 6 of a heart in accordance with one or more examples. While the coaptation device 400 is described with respect to the mitral valve 6, coaptation devices may additionally or alternatively be implanted for repair of any other valve (e.g., the tricuspid valve) and/or other anatomy of the heart (e.g., the papillary muscles). The coaptation device 400 may be used to repair a heart valve, such as a mitral valve 6, and improve functionality thereof. For example, the coaptation device 400 may be used to reduce the degree of mitral regurgitation in patients suffering from mitral regurgitation caused by, for example, midsegment prolapse of valve leaflets as a result of degenerative mitral valve disease. In order to repair such a valve, the coaptation device 400 may comprise one or more tissue anchoring elements 402, such as suture-knot-type tissue anchoring elements 402, in a prolapsed valve leaflet. As described in detail below, such procedures may be implemented on a beating heart.

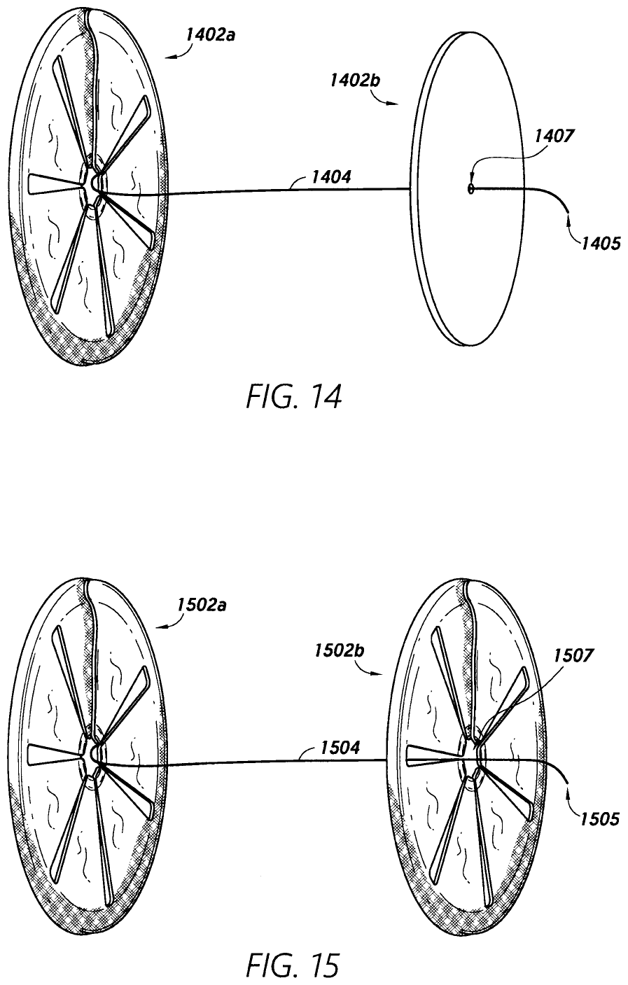

The coaptation device 400 may comprise one or more anchoring elements 402 (e.g., a first anchoring element 402a and a second anchoring element 402b) and/or one or more sutures 404. Anchoring elements may include suture anchoring elements (see, e.g., FIGS. 7-12), “sawtooth” anchoring elements (see, e.g., FIGS. 13A-13C), disc anchoring elements (see, e.g., FIG. 14), balloon anchoring elements, and/or any other device configured to anchor a suture to a valve leaflet. The one or more sutures 404 may be configured to connect multiple anchoring elements 402 or may connect to a single anchoring element 402. The one or more sutures 404 may be cinched to apply pressure to the anchoring elements 402.

In some instances, the coaptation device 400 may be delivered to the heart percutaneously. For example, a catheter may be inserted into the right ventricle and may be passed through the septum into the left ventricle 3. Additionally or alternatively, a catheter may be inserted into the left ventricle 3 through, for example, a posterior wall. In some instances, a catheter may be inserted through the tricuspid valve, aortic valve, mitral valve, apex region (transapical), or through any other valve and/or ventricle wall. In the examples shown in FIGS. 4A-1, 4A-2, 4B-1, 4B-2, and 4C, the coaptation device 400 may be configured to repair the mitral valve 6. However, some instances may involve reducing volume of the tricuspid valve or other heart valve or heart anatomy, or may be implanted outside the heart. By delivering the device through the septum and/or posterior wall, there may be a reduced risk of bleeding and open-heart surgery may not be required for implanting the device.

While figures herein may be described with reference to the heart and valve repair, some examples may be configured for delivery to parts of the body other than the heart and may be used for purposes other than valve repair. Moreover, while coaptation devices are shown as being implanted at heart valves, some instances may involve delivering one or more coaptation devices to one or more papillary muscles. For example, a first anchoring element 402a may be inserted into a first papillary muscle and a second anchoring element 402b may be inserted into a second papillary muscle and a suture 404, when tightened, may create pressure at the first and second anchoring elements to move the first and second papillary muscles closer together.

In some instances, each of the one or more sutures 404 may comprise one or more lengths of material and/or may be attached to the anchoring elements 402. Each of the one or more lengths of material may be a cord, string, wire, band, tube, or other similar device. In some instances, the one or more sutures 404 may comprise one or more flexible or rigid mechanisms and may be capable of tensioning (e.g., cinching) to decrease a distance between one or more anchoring elements 402 at a first valve leaflet 61a and one or more anchoring elements 402 at a second valve leaflet 61b. The suture(s) 404 may be configured to be connected to any of the one or more anchoring elements 402 and/or may pass through the anchoring elements 402. In some instances, the suture(s) 404 may be configured to be tensioned and locked into place through use of a locking element or otherwise to lock the one or more anchoring elements 402 in place with respect to each other. The one or more sutures 404 and/or one or more anchoring elements 402 may be passed through a catheter. Each of the one or more anchoring elements 402 may be configured to be passed through a ventricle wall.

As the suture 404 is tensioned, the suture 404 may be configured to apply force to the first anchoring element 402a and/or the second anchoring element 402b to cause the first anchoring element 402a to move towards the second anchoring element 402b and/or to cause the second anchoring element 402b to move towards the first anchoring element 402a. Accordingly, tightening the suture 404 may cause the first leaflet 61a and the second leaflet 61b to move closer together, thereby closing or nearly closing at least a portion the valve 6.

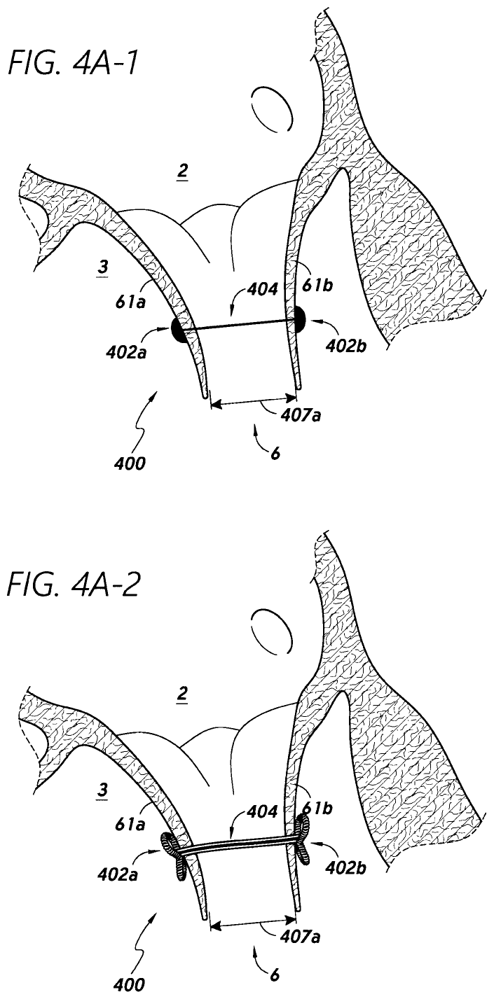

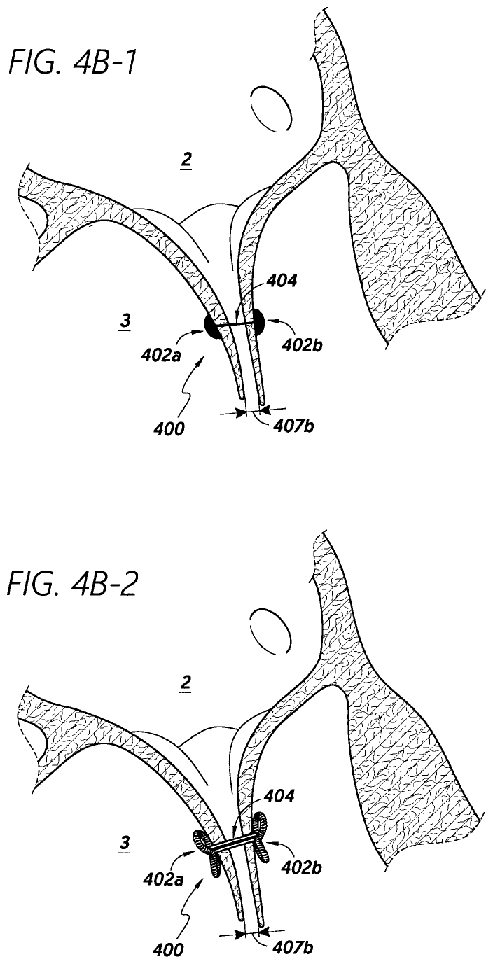

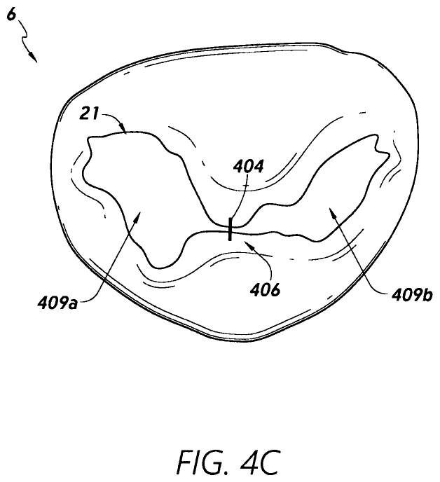

Each anchoring element 402 may be configured to be delivered and/or attached to a leaflet 61 of the valve 6. As shown in FIGS. 4A-1 and 4B-1, a first anchoring element 402a may be situated at a first leaflet 61a (e.g., a posterior leaflet) and a second anchoring element 402b may be positioned at a second leaflet 61b (e.g., an anterior leaflet).

In some examples, the coaptation device 400 may be configured to cause movement of one or more leaflets 61 of the valve 6. The device may be delivered when the valve is in a dilated state shown in FIG. 4A-1. In the dilated state, at least a portion of the valve 6 may be dilated such that a first distance 407a between the first leaflet 61a and the second leaflet 61b is greater than in an undilated state. Accordingly, the suture 404 may have a suitable length such that the suture 404 may extend at least the first distance 407a and/or may be configured to be shortened and/or cinched to decrease the distance between the first leaflet 61a and second leaflet 61b.

As shown in FIG. 4B-1, by cinching the suture 404, the first anchoring element 402a and/or the second anchoring element 402b may be moved closer together and/or the first leaflet 61a and the second leaflet 61b may be moved closer together. In some examples, the device 400 may be configured to cause movement of both the first leaflet 61a and the second leaflet 61b. However, the device 400 may be configured to cause movement of only a single leaflet 61 to reduce distance between the first leaflet 61a and second leaflet 61b. As shown in FIG. 4B-1, the distance between the first leaflet 61a and the second leaflet 61b following cinching of the suture 404 may be a second distance 407b. The second distance 407b may be less than first distance 407a. For example, the second distance 407b may be less than half of the first distance 407a.

In some examples, one or more anchoring elements 402 may be configured to be situated and/or delivered at or near an edge portion of a leaflet 61. This may advantageously allow the device to create maximal approximation of the valve leaflets 61.

In some examples, the coaptation device 400 may be configured to cause movement of one or more leaflets 61 of the valve 6. The device may be delivered when the valve is in a dilated state shown in FIG. 4A-2. In the dilated state, at least a portion of the valve 6 may be dilated such that a first distance 407a between the first leaflet 61a and the second leaflet 61b is greater than in an undilated state. Accordingly, the suture 404 may have a suitable length such that the suture 404 may extend at least the first distance 407a and/or may be configured to be shortened and/or cinched to decrease the distance between the first leaflet 61a and second leaflet 61b.

As shown in FIG. 4B-2, by cinching the suture 404, the first anchoring element 402a and/or the second anchoring element 402b may be moved closer together and/or the first leaflet 61a and the second leaflet 61b may be moved closer together. In some examples, the device 400 may be configured to cause movement of both the first leaflet 61a and the second leaflet 61b. However, the device 400 may be configured to cause movement of only a single leaflet 61 to reduce distance between the first leaflet 61a and second leaflet 61b. As shown in FIG. 4B-2, the distance between the first leaflet 61a and the second leaflet 61b following cinching of the suture 404 may be a second distance 407b. The second distance 407b may be less than first distance 407a. For example, the second distance 407b may be less than half of the first distance 407a.

The coaptation device 400 may comprise one or more anchoring elements 402 (e.g., a first anchoring element 402a and a second anchoring element 402b) and/or one or more sutures 404. Anchoring elements 402 may comprise suture anchoring elements. The one or more sutures 404 may be configured to connect multiple anchoring elements 402 or may connect to a single anchoring element 402. The one or more sutures 404 may be cinched to apply pressure to the anchoring elements 402 and/or to cause a desired amount of valve coaptation. The one or more sutures 404 may be configured to be implemented as tethering sutures and may tether the first anchoring element 402a to the second anchoring element 402b.

In some examples, one or more anchoring elements 402 may be configured to be situated and/or delivered at or near an edge portion of a leaflet 61. This may advantageously allow the device to create maximal approximation of the valve leaflets 61.

FIG. 4C provides an overhead view of the valve 6 with an implanted device comprising a suture 404. As shown, the suture 404 may be configured to significantly reduce an opening of the valve at least a first point 406 of the valve 6, for example at a central region of the valve 6. Blood flow through the valve 6 may be possible following cinching of the suture 404 through other portions of the valve 6 where the device is not placed. For example, as shown in FIG. 4C, the device may be delivered to a central region of the valve and/or may be configured to maintain one or more orifices (e.g., a first orifice 409a and/or a second orifice 409b) on either side of the device to allow flow through the valve 6. While the suture 404 is shown at the central region of the valve 6, the suture 404 and/or one or more anchoring elements (not shown in FIG. 4C) may additionally or alternatively be placed at any other point of the valve 6. In some examples, multiple devices and/or multiple sutures 404 may be delivered to a valve 6. In some cases, each suture 404 may be tethered between two anchoring elements 402. Each anchoring element 402 may be situated at a valve 6 and/or at a leaflet 61 of the valve 6. In some instances, multiple sutures 404 may be configured to be tethered to a common anchor 402. For example, two sutures 404 may be anchored to a first anchoring element 402a at a first leaflet 61a. The two sutures 404 may each be anchored to a second anchoring element 402b at a second leaflet 61b or may be anchored to different anchoring elements 402 at the second leaflet 61b.

Although the procedures described herein are with reference to repairing a cardiac mitral valve or tricuspid valve by the implantation of one or more leaflet anchors and associated cord(s), the methods presented are readily adaptable for various types of tissue, leaflet, and annular repair procedures. The methods described herein, for example, can be performed to selectively approximate two or more portions of tissue to limit a gap between the portions. That is, in general, the methods herein are described with reference to a mitral valve but should not be understood to be limited to procedures involving the mitral valve.

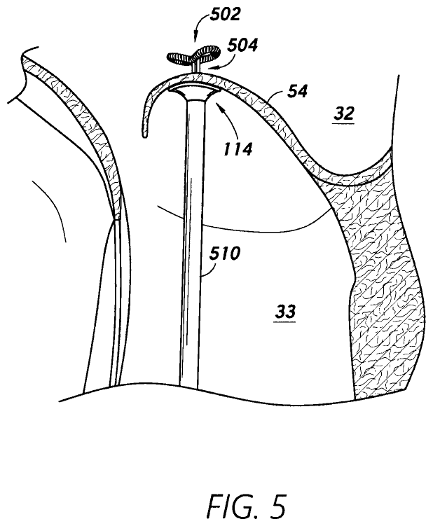

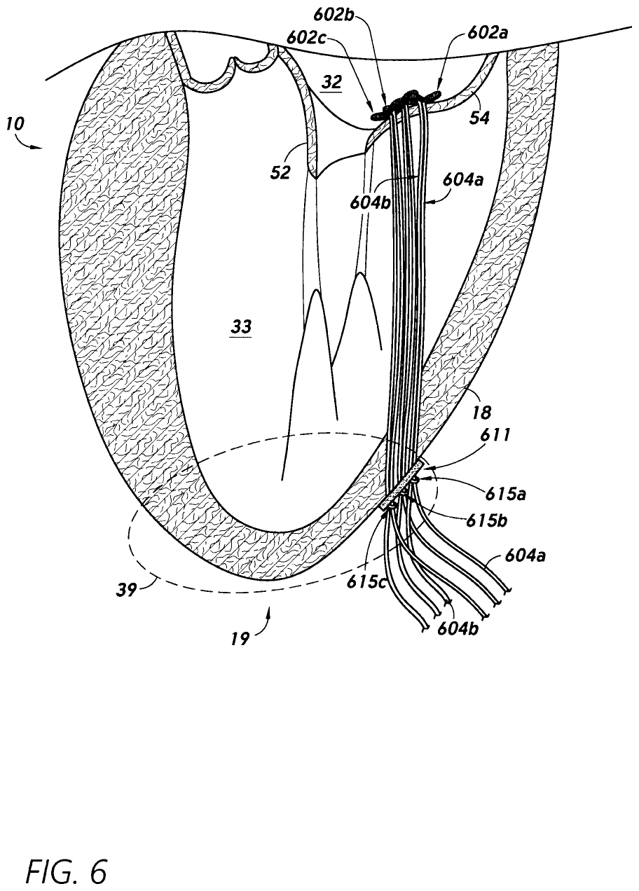

FIG. 5 shows a close-up view of a shaft 510 of a tissue anchor delivery device inserted into a ventricle 33 (e.g., left ventricle) and approximated to a target valve leaflet 54 in connection with a valve-repair procedure in accordance with one or more instances of the present disclosure. For example, the valve may be a mitral valve. The anchor delivery device shaft 510 can be configured to deliver a tissue anchor 502, such as a bulky knot, to the valve leaflet 54. As an example, FIG. 5 shows a valve leaflet 54, which may represent a posterolateral leaflet of a mitral valve. It will be understood that the anchor delivery device shaft 510 can also or alternatively deliver a tissue anchor to the anteromedial mitral valve leaflet. Although the description of FIGS. 5 and 6 below is presented in the context of a mitral valve, it should be understood that the principles disclosed herein are applicable to other valves or biological tissues, such as a tricuspid valve.

With reference to FIGS. 5 and 6, the anchor delivery device shaft 510 can comprise one or more elongate lumens configured to allow delivery of one or more anchors to the valve leaflet 54. The shaft 510 can be configured to facilitate performance of one or more functions, such as grasping, suctioning, irrigating, cutting, suturing, or otherwise engaging a valve leaflet. The distal end, or tip, 114 of the shaft 510 can be configured to contact the mitral valve leaflet 54 without substantially damaging the leaflet to facilitate repair of the valve. For example, during a valve-repair procedure, the shaft 510 and/or a handle coupled to the shaft 510 can be manipulated in such a manner so that the leaflet 54 is contacted with the functional distal portion of the shaft 510 and a repair effectuated.

Echo imaging guidance, such as transesophageal echocardiogram (TEE) (2D and/or 3D), transthoracic echocardiogram (TTE), and/or intracardiac echo (ICE), may be used to assist in the advancement and desired positioning of the anchor delivery device shaft 510 within the ventricle 33. The distal end 114 of the shaft 510 can contact a proximal surface (e.g., underside surface with respect to the illustrated orientation of FIGS. 5 and 6) of the mitral valve leaflet 54, without or substantially without damaging the leaflet 54. For example, the end/tip portion or component 114 can have a relatively blunt form or configuration. The end/tip portion or component 114 can be configured to maintain contact with the proximal side of the valve leaflet 54 as the heart beats to facilitate reliable delivery of the anchor 502 to the target site on the leaflet 54.

In some instances, one or more perforation devices (e.g., needle(s)) can be delivered through one or more working lumens (not shown) of the shaft 510 to the valve leaflet 54 to puncture the valve leaflet 54 and project one or more sutureforms including a plurality of winds of suture about a distal portion of a needle (not shown) into the atrium 32, wherein the one or more sutureforms are deployed to form one or more bulky knot tissue anchors 502. For example, one or more slotted needles can be deployed from the distal end of the shaft 510, thereby puncturing the leaflet 54 and projecting into the atrium 32, wherein each of the one or more slotted needles is configured to be wrapped with a sutureform (e.g., PTFE suture) in a particular configuration. In some instances, one or more pushers and/or hollow guide wires (not shown) may be provided on or at least partially around the one or more needles within the shaft 510, such that the one or more needles may be withdrawn, leaving the pushers and wound sutureforms. When a withdrawal force is applied to the one or more sutureforms using the one or more pushers, the sutureforms may form one or more bulky-knot-type anchors 502, after which the pushers may be withdrawn, leaving the permanent knots 502 to anchor the suture(s) 504 to the leaflet 54.

In some instances, the target site of the valve leaflet 54 may be slowly approached from the ventricle side thereof by advancing the distal end of the shaft 510 along or near to the posterior wall of the ventricle 33 (e.g., left ventricle) without contacting the ventricle wall. Successful targeting and contacting of the target location on the leaflet 54 can depend at least in part on accurate visualization of the shaft 510 and/or tip/end effector 114 throughout the process of advancing the tip 114 to the target site. Generally, echocardiographic equipment may be used to provide the necessary or desired intra-operative visualization of the shaft 510 and/or tip 114.

Once the tip 114 is positioned in the desired position, the distal end of the shaft 510 and the tip 114 may be used to drape, or “tent,” the leaflet 54 to better secure the tip 114 in the desired position. Draping/tenting may advantageously facilitate contact of the tip 114 with the leaflet 54 throughout one or more cardiac cycles, to thereby provide more secure or proper deployment of leaflet anchor(s). The target location may advantageously be located relatively close to the free edge of the target leaflet 54 to minimize the likelihood of undesirable intra-atrial wall deployment of the anchor. Navigation of the tip 114 to the desired location on the underside of the target valve leaflet 54 may be assisted using echo imaging, as described in detail herein. Echo imaging may be relied upon to confirm correct positioning of the tip 114 prior to anchor/knot deployment.

With the shaft 510 positioned against the target leaflet 54, the plunger of the tissue anchor delivery device can be actuated to move the one or more needles and/or one or more pushers disposed within the shaft 510, such that the coiled sutureform portions of the suture anchors 502 slide off the needles. As the plunger is actuated, distal piercing portions of the needles puncture the leaflet 54 and form one or more openings in the leaflet.

As one or more pushers (not shown) within the tissue anchor delivery device shaft 510 are moved distally, distal ends of the pushers may advantageously move and/or push the distal coiled sutureforms (e.g., pre-deployment coiled portions of the suture anchors 502) over the distal ends of the needles and further within the atrium 32 of the heart on a distal side of the leaflet 54, such that the sutureforms extend distally beyond distal ends of the needles. For example, in some instances, at least half a length of a sutureform may be configured to extend beyond the distal end of a needle. The pushers may be configured to press against the sutureforms and/or the needles may be configured to press the sutureforms against the pushers. In some instances, at least three quarters of the length of a sutureform may extend beyond the distal end of a needle. In some instances, an entire coiled sutureform may be configured to extend beyond a distal end of a needle.

After a sutureform has been pushed off and/or removed from a needle, pulling one or more of the suture tail(s) 504 (e.g., suture strands extending from the coiled portion of the suture) associated with the tissue anchor 502 proximally can cause the sutureform to form a bulky knot anchor 502. For example, the bulky knot suture anchor 502 may be formed by approximating opposite ends of the coils of the sutureform towards each other to form one or more loops. After the sutureform has been formed into the bulky knot 502, the delivery device can be withdrawn proximally, leaving the tissue anchor 502 disposed on the distal atrial side of the leaflet 54. In some instances, two suture tails 504 for each bulky knot 502 may extend from the proximal/ventricle side 33 of the leaflet 54 and out of the heart 1. For example, the delivery device shaft 510 can be slid/withdrawn over the suture tail(s) 504.

The shaft 510 may allow for the placement of artificial chordae at one or more leaflets of valves of the heart through a relatively small thoracotomy and/or a relatively small puncture (e.g., a fourteen French puncture) near an apex region of a ventricle (e.g., the left ventricle). Artificial chordae may be configured to be deployed with a bulky knot at an atrium 32 side of the valve leaflet 54 with the chordae extending toward the access site on the ventricle 33. In some instances, several bulky knots and/or several chordae can be placed in a first leaflet 54 (e.g., a posterior leaflet) and/or lengths of the chordae can be titrated to restore proper coaptation to the prolapsing segment. The artificial chordae can then be tied to the proper length over a pledget on the outside of the heart. This process may advantageously result in reduction and/or elimination of mitral regurgitation without the need for cardiopulmonary bypass, thus reducing the risk to the patient and/or expediting recovery.

Because a valve may require multiple bulky knots and/or artificial chordae, examples described herein provide systems and methods for delivering multiple bulky knots and/or artificial chordae using a single delivery device (e.g., a single shaft 510 and/or needle). This may advantageously minimize cost of goods, packaging, medical waste, and/or clutter in the operating space, and/or may increase convenience for surgeons and/or staff.

FIG. 6 shows a cutaway view of multiple deployed leaflet anchors 602a, 602b, 602c in accordance with one or more instances of the present disclosure. While FIG. 6 shows three deployed leaflet anchors 602, any number of leaflet anchors may be deployed. A first leaflet anchor 602a, second leaflet anchor 602b, and/or third leaflet anchor 602c may be delivered sequentially via a delivery shaft. In some instances, the leaflet anchors 602 may each be delivered via the same delivery shaft without requiring removal of the delivery shaft from the body and/or the ventricle 33. In other words, the delivery shaft may be configured to be delivered into the ventricle 33 and/or into contact with the leaflet 54 and/or to deploy multiple leaflet anchors 602 before removal of the delivery shaft 510 from the ventricle 33 and/or leaflet 54.

The suture tails 604 coupled to the anchors 602 may be secured at the desired tension using a pledget 611 and/or other suture-fixing/locking device and/or mechanism on the outside of the heart through which the suture tails 604 may run. For example, a first suture tail 604a and/or a second suture tail 604b of a first leaflet anchor 602a may form a first knot 615a to secure the first leaflet anchor 602a to the pledget 611. Additional knots (e.g., 615b and/or 615c) may be formed to secure and/or cinch the second anchoring element 602b and/or third anchoring element 602c in place. In some instances, the pledget 611 may be configured to be situated at or near an apex 19 and/or an apex region 39 of the heart. The pledget 611 may be at least partially situated outside the ventricle 33. While multiple leaflet anchors 602 may be secured using a single pledget 611 as shown in FIG. 6, multiple pledgets 611 may alternatively be used. For example, each knot 615 may secure to a separate pledget 611. The knots 615 and/or other suture fixation mechanism(s) or device(s) may be implemented to hold the sutures at the desired tension and/or to the pledget 611. With the suture tail(s) 604 fixed to the ventricle wall 18 and/or apex region 39, a ventricular portion of the suture tail(s) 604 (e.g., the portion of the suture tail 604 that is within the ventricle 33) may advantageously function as replacement leaflet cords (e.g., chordae tendineae) that may be configured to tether the target leaflet 54 in a desired manner.

In certain instances, the pledget 611 may be a low-porosity and/or relatively stiff pledget. Such a pledget 611 may advantageously allow for the desired tension of the suture tails 604 to be sustained over an extended post-operative period of time. In some instances, suture tying and/or fixation may be implemented using one or more soft tissue retractors and/or right-angle clamps, which may have rubber shods associated therewith to reduce the risk of damage to the replacement cords.

In certain implementations, testing of location and/or tension of the anchor 602 and/or suture tail(s) 604 may be performed by gently tensioning the suture tails until leaflet motion is felt and/or observed. Echo imaging technology may be used to view and verify the anchor placement and resulting leaflet function. The steps and processes outlined above for placing one or more suture-knot-type tissue anchors may be repeated as necessary until the desired number of anchors have been implanted on the target valve leaflet. In some implementations, tension adjustment in the suture tail(s)/cord(s) associated with multiple leaflet anchors may be performed simultaneously. The appropriate number of leaflet anchors may advantageously be determined to produce the desired coaptation of the target valve leaflets 54, 52. All deployed leaflet anchors may advantageously be below the surface of coaptation. With respect to posterior mitral valve leaflet repair, the anterior leaflet may advantageously touch the posterior leaflet basal to the leaflet anchor(s). The pledget 611 may be drawn against the epicardial surface, and all the suture tails/cords 604 may be inserted through a tourniquet so that all cords can be tension to the desired effective coaptation together.

In some implementations, one or more leaflet anchors may be deployed in each of the mitral valve leaflets, and/or sutures/cords coupled to separate leaflets may be secured together in the heart by tying them together with knots and/or by another suitable attachment device, creating an edge-to-edge repair to decrease the septal-lateral distance of the mitral valve orifice.

It may be desirable to advance the shaft within the ventricle 33 in such a way as to avoid traversing areas populated by papillary muscles and/or associated chordae tendineae to avoid entanglement therewith. In order to facilitate or ensure avoidance of such anatomy, imaging technology may advantageously be implemented to provide at least partial visibility of the shaft within the ventricle 33, as well as certain anatomical features within the ventricle. With respect to visibility of the shaft in the ventricle 33, echogenic characteristics of the shaft can affect the visibility thereof using echo imaging modalities. Therefore, a shaft having relatively high echogenicity as described in detail herein may advantageously allow for more accurate and/or simplified advancement of the shaft, as well as placement of the tip 114 at the target implantation site at the valve leaflet 54 (e.g., an anterior or posterior leaflet of a mitral valve). In some implementations, hybrid imaging technologies may be used, wherein echo imaging is used in combination with a different imaging modality. Multi-imaging modalities may provide improved visibility of anatomical and/or delivery system components.

### Anchoring Devices

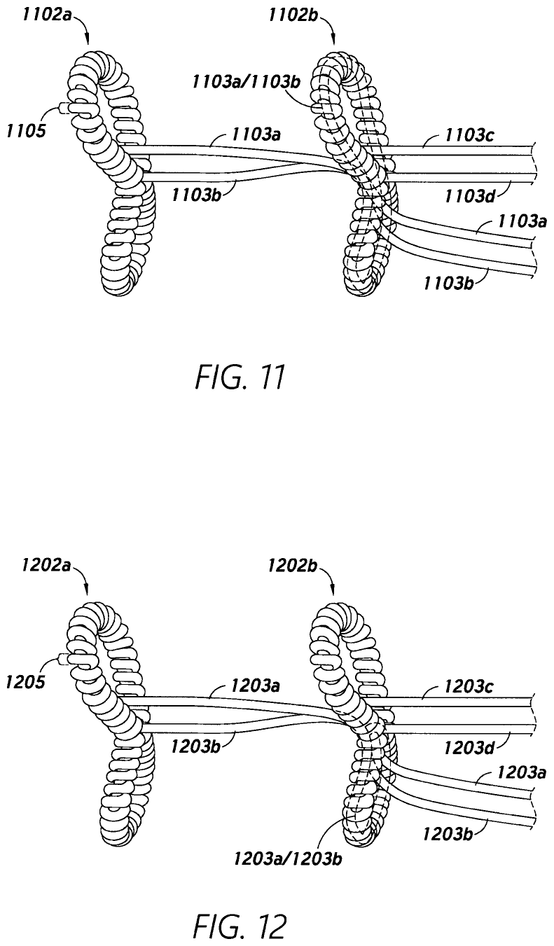

FIG. 7 shows a coaptation device comprising a first anchoring element 702a and a second anchoring element 702b in accordance with one or more examples. In some examples, one or more anchoring elements 702 may comprise one or more sutures formed into a sutureform. An anchoring element 702 formed at least partially of a suture may be referred to herein as a “suture anchor” or “suture anchoring element.” A sutureform may comprise an at least partially coiled suture comprising one or more coils 705. The sutureform may be configured to be formed into a knot (e.g., a “bulky knot”) as shown in FIGS. 11 and 12. One or more tail portions 703 of an anchoring element 702 may be configured to extend from and/or may be an extension (e.g., a free end) of the sutureform and/or knot to attach to, interconnect with, and/or interlock with another sutureform, knot, and/or other type of anchoring element 702. Accordingly, one or more tail portions 703 may form tethering sutures between multiple anchoring elements 702.

In some examples, a suture forming one or more anchoring elements 702 may also serve as a tethering suture between multiple anchoring elements 702. For example, a first (e.g., distal) anchoring element 702a may be at least partially formed of a suture having two tail portions 703 (e.g., a first tail portion 703a and a second tail portion 703b) which may be configured to attach to, extend into, and/or secure to a second (e.g., proximal) anchoring element 702b. One or more tail portions 703 extending from the first anchoring element 702a may be configured to fit through one or more coils 705 of the second anchoring element 702b such that at least a portion of the second anchoring element 702b may be wrapped around one or the one or more tail portions 703 extending from the first anchoring element 702a. The first anchoring element 702a and/or second anchoring element 702b may be coiled to form internal lumens through the one or more coils/windings 705 of the first anchoring element 702a and/or second anchoring element 702b. The second anchoring element 702b may additionally comprise one or more tail portions 703 (e.g., a third tail portion 703c and/or a fourth tail portion 703d) which may be configured to extend out of the sutureform and/or knot of the second anchoring element 702b. In some examples, the one or more tail portions 703 extending from the first anchoring element 702a (e.g., the first tail portion 703a and/or second tail portion 703b) may at least partially form the second anchoring element 702b. In other words, the first anchoring element 702a and the second anchoring element 702b may be formed from a common set of one or more sutures (e.g., a single suture). In such cases, less than four tail portions 703 (e.g., two tail portions 703) may extend from the second anchoring element 702b.

In the example shown in FIG. 7, the first tail portion 703a may extend from a distal end 707a of the first anchoring element 702a and/or may be configured to pass along the first anchoring element 702a external to all of the one or more coils 705 of the first anchoring element 702a to reach the second anchoring element 702b. The second tail portion 703b may extend directly from a proximal end 707b of the first anchoring element 702a to reach the second anchoring element 702b. Each of the first tail portion 703a and the second tail portion 703b may be configured to enter a distal end 709a of the second anchoring element 702b and/or pass entirely through an internal lumen of the second anchoring element 702b to exit a proximal end 709b of the second anchoring element 702b.

FIG. 8 shows another coaptation device comprising a first anchoring element 802a and a second anchoring element 802b in accordance with one or more examples. In some examples, a first tail portion 803a of the first anchoring element 802a may be configured to extend from a distal end 807a of the first anchoring element 802a and/or pass along at least a portion of the first anchoring element 802a external to at least one of one or more coils 805 of the first anchoring element 802a. The first tail portion 803a may be configured to pass between coils 805 of the first anchoring element 802a. The first tail portion 803a may be configured to interlock with a second tail portion 803b at a midpoint of the first anchoring element 802a. In some instances, the second tail portion 803b may be configured to exit from an internal lumen of the first anchoring element 802a at a midpoint of the first anchoring element 802a (e.g., between two coils 805 of the first anchoring element 802a) and/or may interlock with the first tail portion 803a. For example, the first tail portion 803a may be twisted with the second tail portion 803b to form a secure attachment between the first tail portion 803a and the second tail portion 803b at a midpoint of the first anchoring element 802a. In some instances, the first tail portion 803a and/or the second tail portion 803b may be configured to form one or more coils 805 of the first anchoring element 802a and/or of the second anchoring element 802b. The second tail portion 803b may be configured to pass along an exterior of at least a portion of the first anchoring element 802a (e.g., along one or more coils 805 at or near a proximal end 807b of the first anchoring element 802a) at or near the proximal end 807b of the first anchoring element 802a to reach the second anchoring element 802b. The second anchoring element 802b may additionally comprise one or more tail portions 803 (e.g., a third tail portion 803c and/or a fourth tail portion 803d) which may be configured to extend out of the sutureform and/or knot of the second anchoring element 802b. In some instances, the first tail portion 803a and/or the second tail portion 803b may be configured to be twisted with each other and/or with one or more coils 805 of the first anchoring element 802a to form a secure attachment to each other and/or to the coils 805 of the first anchoring element 802a.

In some instances, the first tail portion 803a and/or the second tail portion 803b may be configured to pass between coils 805 of the second anchoring element 802b to enter an internal lumen of the second anchoring element 802b. The first tail portion 803a and/or the second tail portion 803b may be configured to pass within the internal lumen of the second anchoring element 802b and/or exit the internal lumen at a proximal end 809b of the second anchoring element 802b. The internal lumen of the second anchoring element 802b may also open at a distal end 809a. The third tail portion 803c and/or the fourth tail portion 803d may be configured to exit the internal lumen of the second anchoring element 802b between coils 805 of the second anchoring element 802b and/or at a midpoint of the second anchoring element 802b and/or may be configured to extend along an exterior of a proximal portion of the second anchoring element 802b.

In some examples, a first tail portion 803a of the second anchoring element 802b may be configured to extend from the distal end 809a of the second anchoring element 802b and/or pass along the second anchoring element 802b external to at least one of one or more coils 805 of the second anchoring element 802b. The first tail portion 803a may be configured to pass between coils 805 of the second anchoring element 802b. The first tail portion 803a may be configured to interlock with a second tail portion 803b at a midpoint of the second anchoring element 802b. In some instances, the second tail portion 803b may be configured to exit from an internal lumen of the second anchoring element 802b at a midpoint of the second anchoring element 802b (e.g., between two coils 805 of the second anchoring element 802b) and/or may interlock with the first tail portion 803a. For example, the first tail portion 803a may be twisted with the second tail portion 803b to form a secure attachment between the first tail portion 803a and the second tail portion 803b at a midpoint of the second anchoring element 802b. The second tail portion 803b may be configured to pass along an exterior of the second anchoring element 802b at or near the proximal end 809b of the second anchoring element 802b and/or may be configured to extend out of the patient's body and/or may otherwise be accessible to a surgeon. In some instances, the first tail portion 803a and/or the second tail portion 803b may be configured to be twisted with one or more coils 805 of the second anchoring element 802b to form a secure attachment to the coils 805 of the second anchoring element 802b.

FIG. 9 shows another coaptation device comprising a first anchoring element 902a and a second anchoring element 902b in accordance with one or more examples. A first tail portion 903a of the first anchoring element 902a may be configured to extend from a distal end 907a of the first anchoring element 902a and/or along an exterior surface of the first anchoring element 902a and/or may pass between one or more coils 905 of the first anchoring element 902a to secure to a second tail portion 903b of the first anchoring element 902a. The second tail portion 903b may be configured to extend from a proximal end 907b of the first anchoring element 902a, to interlock with the first tail portion 903a, and/or to exit from a midpoint of the first anchoring element 902a and/or to extend at least partially along a proximal portion of the first anchoring element 902a.

In some instances, the first tail portion 903a and/or the second tail portion 903b extending from and/or at least partially forming the first anchoring element 902a may be configured to enter an internal lumen of the second anchoring element 902b. The first tail portion 903a may be configured to enter the internal lumen of the second anchoring element 902b at a distal end 909a of the second anchoring element 902b and/or the second tail portion 903b may be configured to enter the internal lumen of the second anchoring element 902b at a proximal end 909b of the second anchoring element 902b. The first tail portion 903a and/or the second tail portion 903b may be configured to pass within the internal lumen of the second anchoring element 902b and/or pass between coils 905 of the second anchoring element 902b to exit the second anchoring element 902b. In some instances, the first tail portion 903a and/or the second tail portion 903b may be configured to pass between coils 905 at a midpoint of the second anchoring element 902b, as shown in FIG. 9.

The second anchoring element 902b may be at least partially formed by a third tail portion 903c and/or a fourth tail portion 903d. The third tail portion 903c of the second anchoring element 902b may be configured to extend from a distal end 909a of the second anchoring element 902b and/or along an exterior surface of the second anchoring element 902b and/or may pass between one or more coils 905 of the second anchoring element 902b to secure to the fourth tail portion 903d of the second anchoring element 902b. The fourth tail portion 903d may be configured to extend from a proximal end 909b of the second anchoring element 902b, to interlock with the third tail portion 903c, and/or to exit from a midpoint of the second anchoring element 902b and/or to extend at least partially along an exterior surface of a proximal portion of the second anchoring element 902b.

FIG. 10 shows another coaptation device comprising a first anchoring element 1002a and a second anchoring element 1002b in accordance with one or more examples. A first tail portion 1003a of the first anchoring element 1002a may be configured to extend from a distal end 1007a of the first anchoring element 1002 and/or along an exterior surface of the first anchoring element 1002a and/or may pass between one or more coils 1005 of the first anchoring element 1002a to secure to a second tail portion 1003b of the first anchoring element 1002a. The second tail portion 1003b may be configured to extend from a proximal end 1007b of the first anchoring element 1002a, to interlock with the first tail portion 1003a, and/or to exit from a midpoint of the first anchoring element 1002a and/or to extend at least partially along a proximal portion of the first anchoring element 1002a.

In some instances, the first tail portion 1003a and/or the second tail portion 1003b extending from the first anchoring element 1002a may be configured to pass between coils 1005 of the second anchoring element 1002b to enter an internal lumen of the second anchoring element 602b. The first tail portion 1003a and/or the second tail portion 1003b may be configured to pass within the internal lumen of the second anchoring element 1002b and/or exit the internal lumen at a distal end 1009a and/or a proximal end 1009b of the second anchoring element 1002b. For example, the first tail portion 1003a may be configured to exit the second anchoring element 1002b at the distal end 1009a of the second anchoring element 1002b and the second tail portion 1003b may be configured to exit the second anchoring element 1002b at the proximal end 1009b of the second anchoring element 1002b.

The second anchoring element 1002b may be at least partially formed by a third tail portion 1003c and/or a fourth tail portion 1003d. The third tail portion 1003c of the second anchoring element 1002b may be configured to extend from a distal end 1009a of the second anchoring element 1002b and/or along an exterior surface of the second anchoring element 1002b and/or may pass between one or more coils 1005 of the second anchoring element 1002b to secure to the fourth tail portion 1003d of the second anchoring element 1002b. The fourth tail portion 1003d may be configured to extend from a proximal end 1009b of the second anchoring element 1002b, to interlock with the third tail portion 1003c, and/or to exit from a midpoint of the second anchoring element 1002b and/or to extend at least partially along an exterior surface of a proximal portion of the second anchoring element 1002b.

FIGS. 11 and 12 illustrate coaptation devices comprising two anchoring elements forming knots (e.g., bulky knots). In some examples, each of the suture anchoring elements in FIGS. 7-10 may be configured to form a knot. For example, a sutureform may be configured to form a knot when the sutureform is removed from a from a delivery device (e.g., a needle). The knots formed by the sutureforms may create one or more openings and/or loop portions. For example, a knot may form a figure-8 pattern comprising two interlocking loop portions, as shown in FIGS. 11 and 12. One or more tail portions 1103 of the first anchoring element 1102a may be configured to extend through the one or more openings formed in the second anchoring element 1102b and/or may pass through one or more coils 1105 of the second anchoring element 1102b. In some instances, one or more tail portions 1103 from the first anchoring element 1102a (e.g., the first tail portion 1103a and/or second tail portion 1103b) may be configured to form a tether between the first anchoring element 1102a and the second anchoring element 1102b. Accordingly, when the one or more tail portions 1103 of the first anchoring element 1102a are cinched, the one or more tail portions 1103 may be configured to reduce the distance between the first anchoring element 1102a and the second anchoring element 1102b. The second anchoring element 1102b may be at least partially formed by a third tail portion 1103c and/or a fourth tail portion 1103d.

In some instances, additional knots and/or the first anchoring element 1102a and/or the second anchoring element 1102b may be advanced down one or more tail portions 1103 to lock the one or more tail portions 1103 with respect to each other and/or to lock the first anchoring element 1102a and the second anchoring element 1102b with respect to each other. One or more fasteners and/or suture locking devices may be additionally or alternatively used to lock the tail portions 1103 and/or anchoring elements 1102 in place. In some instances, friction from the various sutures forming the first anchoring element 1102a, second anchoring element 1102b, and/or tail portions may be sufficient to lock the first anchoring element 1102a and the second anchoring element 1102b relative to each other without the need for additional knots and/or other fastening devices.

As shown in FIG. 12, one or more tail portions 1203 of a first anchoring element 1202a may be configured to pass through only a portion of the coils/windings 1205 of the second anchoring element 1202b and/or may not pass through any coils/windings 1205 of the second anchoring element 1202b. For example, a first tail portion 1203a and/or a second tail portion 1203b may be configured to pass through only one loop (e.g., half) of a figure-8 bulky knot, as shown in FIG. 12. One or more tail portions 1203 of the first anchoring element 1202a may enter and/or exit any point of the second anchoring element 1202b. In some instances, one or more tail portions 1203 from the first anchoring element 1202a (e.g., the first tail portion 1203a and/or second tail portion 1203b) may be configured to form a tether between the first anchoring element 1202a and the second anchoring element 1202b. Accordingly, when the one or more tail portions 1203 of the first anchoring element 1202a are cinched, the one or more tail portions 1203 may be configured to reduce the distance between the first anchoring element 1202a and the second anchoring element 1202b. The second anchoring element 1202b may be at least partially formed by a third tail portion 1203c and/or a fourth tail portion 1203d.

FIGS. 13A-11C illustrate an example anchoring element 1302 which may be delivered to one or more valve leaflets in accordance with one or more examples. In some examples, an anchoring element 1302 may be configured to form and/or to be formed into a circular shape as shown in FIG. 13A. The anchoring element 1302 may comprise one or more appendages 1320 extending from a base portion 1324. In some instances, the one or more appendages 1320 may be configured to extend from a base portion 1324 of the anchoring element 1302. The one or more appendages 1320 may be separated from each other by one or more gaps 1322. In some instances, the one or more appendages 1320 may have generally triangular forms in which a width of an appendage 1320 decreases as the appendage 1320 extends from the base portion 1324. In this way, the shapes of the one or more appendages 1320 may allow the base portion 1324 to be bent and/or otherwise shaped into a circular form without the one or more appendages impeding each other.

The one or more appendages 1320 may comprise folds and/or may have cavities 1321 and or other openings to allow one or more sutures 1304 to pass through the appendage 1320, as shown in FIG. 13B. Each of the appendages 1320 may be at least partially hollow at or near an end portion of the appendage 1320 to allow the suture 1304 to fit through the end portion of the appendage 1320 to cinch the anchoring element 1302. The suture 1304 may have a free end 1305 which may be configured to pass through and/or attach to another anchor to form a tether between the anchoring element 1302 and the other anchor. In some examples, the free end 1305 of the suture may be accessible to a surgeon and/or may be pulled to cinch the anchoring element 1302 and/or to move one or more tethered anchoring elements 1302 closer together.

In some examples, the anchoring element 1302 may be configured to form a non-continuous circular shape and/or may comprise multiple end portions 1303 (e.g., at the base portion 1324) which may be at least partially overlapped when in the circular shape. For example, the anchoring element 1302 may comprise a length of cloth and/or other material that may be formed into a circular and/or other shape through use of a shaping device 1326 which may be fit into the base portion 1324 of the anchor. The shaping device 1326 may comprise one or more end portions 1327 (e.g., a first end portion 1327a and/or a second end portion 1327b) which may each be configured to be situated at or near an end portion 1303 of the anchoring element 1302. The first end portion 1327a of the shaping device 1326 may be configured to at least partially overlap with the second end portion 1327b of the shaping device 1326.

An example shaping device 1326 is shown in FIG. 13C. The shaping device 1326 may be configured to form the anchoring element 1302 to any desired shape. In the example shown in FIG. 13C, the shaping device 1326 may be configured to form a non-continuous circular shape in which the first end portion 1327a and the second end portion 1327b of the shaping device 1326 do not connect and/or may be configured to at least partially overlap. By allowing the first end portion 1327a and the second end portion 1327 to at least partially overlap, the shaping device 1326 may be configured to maintain a generally circular shape in spite of pulling force applied by the suture 1304. The shaping device 1326 may be configured to form a consistent outer diameter which may be stable when pressure is applied to the suture 1304.

The anchoring element 1302 may similarly be configured to at least partially overlap in conformance with the shape of the shaping device 1326. The shaping device 1326 may be configured to fit at least partially within the base portion 1324 of the anchoring element 1302. The first end portion 1327a and/or the second end portion 1327b may comprise a notch and/or similar mechanism to prevent the shaping device 1326 from being dislodged from the anchoring element 1302. For example, each end portion 1327 may have a profile which may be too large to fit into the base portion 1324 of the anchoring element 1302. In this way, the end portion 1327 may be prevented from being pulled into the base portion 1324 of the anchoring element 1302. In some examples, the shaping device 1326 may be at least partially composed of a shape-memory alloy (e.g., Nitinol).

As shown in FIG. 13B, the anchoring element 1302 may be configured to form a “sawtooth” structure in which the one or more appendages 1320 form “teeth” and/or extensions from the base portion 1324 of the anchoring element 1302. When the suture 1304 is cinched, each of the appendages 1320 may extend radially inwards toward a center opening 1325 of the anchoring element 1302 in the circular form shown in FIG. 13A. The one or more appendages 1320 may form triangular-shaped gaps 1322 to allow for cinching of the anchoring element 1302 to form the circular shape shown in FIG. 13A. Moreover, the gaps 1322 between the appendages 1320 may allow the anchoring element 1302 to be at least partially overlapped as necessary to form a desired shape and/or profile. In some instances, the anchoring element 1302 may be configured to be at least partially flattened/compressed to allow the anchoring element 1302 to fit within a needle and/or other delivery device having a profile that may be smaller than the anchoring element 1302 in the expanded/circular shape shown in FIG. 13A. At least some portions of the anchoring element 1302 may be configured to be at least partially compressed. In some examples, the anchoring element 1302 may be at least partially formed of cloth and/or other flexible and/or elastic material.

The suture 1304 may be configured to fit within one or more of the appendages 1320 and/or may be configured to be cinched to cause compression of the anchoring element 1302 into the circular shape shown in FIG. 13A. In some examples, one end of the suture 1304 may be formed into a circular (e.g., lasso) shape and/or may comprise a mechanism to secure the end at the anchoring element 1302. For example, a first end of the suture 1304 may be configured to form a loop and at least a portion of the suture 1304 may be configured to feed through the loop to secure the suture 1304 in place at the anchoring element 1302.

FIGS. 14, 15, and 16 illustrate example coaptation devices comprising multiple anchoring elements 1402 (e.g., a first anchoring element 1402a and/or a second anchoring element 1402b) in accordance with some examples. In some examples, a first (e.g., distal) anchoring element 1402a may be at least partially different from a second (e.g., proximal) anchoring element 1402b. For example, a first anchoring element 1402a may comprise a sawtooth design (see, e.g., FIGS. 13A and 13B) while a second anchoring element 1402b may comprise a disc and/or other type of anchor. The first anchoring element 1402a and the second anchoring element 1402b may be joined by a suture 1404 which may be attached to and/or extend from the first anchoring element 1402a and/or may pass through and/or attach to the second anchoring element 1402b (e.g., through an aperture 1407 of the second anchoring element 1402b). The suture 1404 may comprise a free end 1405 which may be formed into a knot and/or may otherwise be configured to secure to the second anchoring element 1402b and/or to secure a desired tension of the suture 1404.

As shown in FIG. 15, a first anchoring element 1502a and a second anchoring element 1502b may have a similar/identical design and/or may each have a sawtooth design. The first anchoring element 1502a and the second anchoring element 1502b may be joined by a suture 1504 which may be attached to and/or extend from the first anchoring element 1502a and/or may pass through and/or attach to the second anchoring element 1502b (e.g., through an opening of the second anchoring element 1502b). The suture 1504 may comprise a free end 1505 configured to extend through an aperture 1507 of the second anchoring element 1502b. The free end 1505 may be formed into a knot and/or may otherwise be configured to secure to the second anchoring element 1502b and/or to secure a desired tension of the suture 1504.

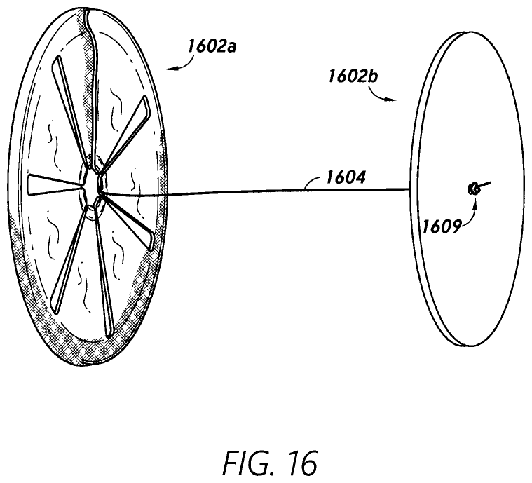

As shown in FIG. 16, the suture 1604 may be configured to be formed into a knot 1609 and/or may be configured to interact with a locking mechanism at or near the second anchoring element 1602b to prevent the suture 1604 from slipping through an aperture of the second anchoring element 1602b and/or to apply pressure at the first anchoring element 1602a and/or the second anchoring element 1602b. In some instances, a suture locking mechanism may be utilized to lock the suture 1604 at the second anchoring element 1602b and/or at the first anchoring element 1602a. For example, a clip formed of titanium and/or other material may be used to lock the suture 1604 at the second anchoring element 1602b. In this way, the first anchoring element 1602a and the second anchoring element 1602b may be locked in place relative to each other.

The first anchoring element 1602a and/or the second anchoring element 1602b may comprise one or more apertures and/or openings at a central and/or other region of the anchoring element 1602 to allow the suture 1604 to pass through the anchoring element 1602. In some instances, the first anchoring element 1602a or and/or the second anchoring element 1602b may be at least partially flexible, elastic, and/or compressible to be able to be delivered via a low-profile needle and/or other delivery device.

The second anchoring element 1602b may be any suitable anchoring element 1602 configured to apply pressure to a valve leaflet. In some instances, the second anchoring element may comprise a pledget and/or other device having a shape configured to distribute load across a portion of a valve leaflet. The first anchoring element 1602a may be configured to be delivered through a needle and/or other similar device. In some instances, the first anchoring element 1602a and/or second anchoring element 1602b may be delivered through one or more valve leaflets prior to being removed from a delivery device (e.g., a needle). The second anchoring element 1602b may be delivered by any suitable means and in some cases may be delivered via a tube and/or similar device. In some examples, the second anchoring element 1602b may not be passed through a leaflet prior to delivery at the valve leaflet. For example, the second anchoring element 1602b may be delivered through a ventricle wall and at a proximal leaflet with respect to the ventricle wall. The second anchoring element 1602b may be configured to be passed over at least a portion of the suture 1604. Once the second anchoring element 1602b is in place, the suture 1604 may be cinched and locked to create a desired amount of coaptation of one or more valve leaflets. In some instances, the first anchoring element 1602a and/or the second anchoring element 1602b may have any suitable size. For example, the first anchoring element 1602a and/or the second anchoring element 1602b may be approximately five millimeters in diameter.

### Coaptation Device Delivery Systems

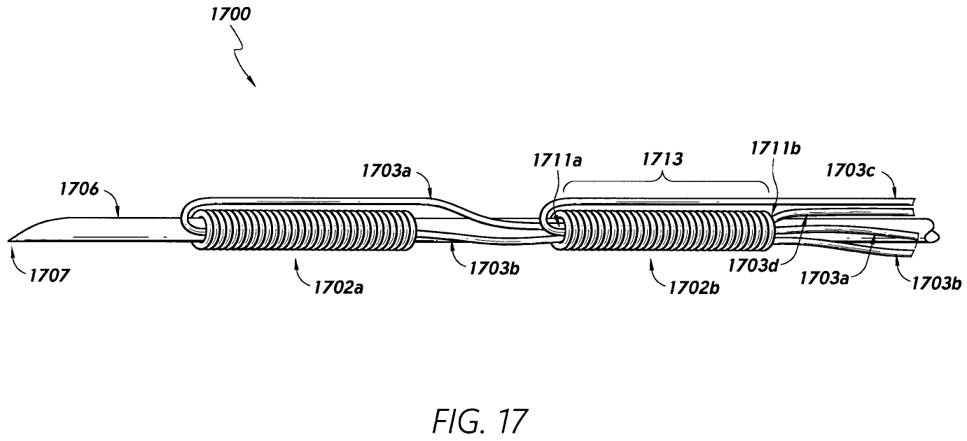

FIG. 17 illustrates an example delivery system 1700 for delivering one or more anchoring elements 1702 to a valve leaflet in accordance with some examples. In the example shown in FIG. 17, two sutureform anchoring elements 1702 (e.g., a first suture anchoring element 1702a and a second suture anchoring element 1702b) may be at least partially wrapped around a needle 1706 or similar device comprising a pointed tip 1707 configured to penetrate and/or pass through one or more valve leaflets. The needle 1706 may be configured to carry the first (e.g., distal) anchoring element 1702a and/or the second (e.g., proximal) anchoring element 1702b. The one or more anchoring elements 1702 may be configured to be delivered simultaneously or sequentially. In some examples, the first anchoring element 1702a and/or the second anchoring element 1702b may be configured to form a knot (e.g., a bulky knot) when removed from the needle 1706. In some cases, a pusher or similar device may be configured to push one or more of the anchoring elements 1702 off of the needle 1706. One or more tail portions 1703 (e.g., a first tail portion 1703a and/or a second tail portion 1703b) of the first anchoring element 1702a may be configured to pass through one or more coils of the second anchoring element 1702b.

In some instances, the first anchoring element 1702a may comprise one or more sutures having one or more suture tails 1703 (e.g., a first suture tail 1703a and a second suture tail 1703b). The first suture tail 1703a and/or the second suture tail 1703b may be configured to extend at least partially through one or more coils/windings of the second anchoring element 1702b. For example, the first suture tail 1703a and/or the second suture tail 1703b may pass entirely through an internal lumen of the second anchoring element 1702b between the coils/windings of the second anchoring element 1702b, as shown in FIG. 17. The first suture tail 1703a and/or the second suture tail 1703b may be situated at least partially between the coils/windings of the second anchoring element 1702b and the delivery device 1706 while the second anchoring element 1702b is attached to and/or wrapped around the delivery device 1706. The first suture tail 1703a and/or second suture tail 1703b may be configured to extend out of the second anchoring element 1702b and/or out of the ventricle and may be accessible to a surgeon. When the first suture tail 1703a and/or second suture tail 1703b is/are pulled, the coils/windings of the second anchoring element 1702b may be configured to circularize to form a knot (e.g., a figure-8/bulky knot, see, e.g., FIG. 11). The first suture tail 1703a and/or the second suture tail 1703b may remain within the coils/windings of the second anchoring element 1702b after the second anchoring element 1702b forms the knot (see, e.g., FIG. 11).

The first suture tail 1703a and/or second suture tail 1703b may be configured to enter the second anchoring element 1702b (e.g., pass through one or more coils/windings of the second anchoring element 1702b) at one or more end portions 1711a, 1711b of the second anchoring element 1702b and/or a mid-portion 1713 of the second anchoring element 1702b (e.g., between the end portions 1711). In some instances, the first suture tail 1703a may be configured to enter the second anchoring element 1702b at a first end portion 1711a, second end portion 1711b, or a mid-portion 1713 and the second suture tail 1703b may be configured to enter the second anchoring element 1702b at the first end portion 1711a, the second end portion 1711b, or at the mid-portion 1713.

In some instances, the delivery device 1706 may be configured to be inserted into the heart via a catheter and/or an elongate tube forming at least one internal working lumen. Implementation of a valve-repair procedure utilizing the delivery system 1700 can be performed in conjunction with certain imaging technology designed to provide visibility of the delivery device 1706, catheter, and/or other components of the delivery system 1700 according to a certain imaging modality, such as echo imaging. Generally, when performing a valve-repair procedure utilizing the tissue anchoring element delivery system 1700, the operating physician may advantageously work in concert with an imaging technician, who may coordinate with the physician to facilitate successful execution of the valve-repair procedure.

In addition to the catheter and/or elongate tube, the delivery system 1700 may include a plunger feature, which may be used or actuated to manually deploy the delivery device 1706 and/or to remove the one or more anchoring elements 1702 from the delivery device 1706. The lumen of the catheter and/or elongate tube may be configured to house the delivery device 1706 wrapped at least in part with one or more pre-formed knot sutureform anchors 1702. In some examples, the catheter and/or elongate tube presents a relatively low profile. For example, a catheter may have a diameter of approximately 3 mm or less (e.g., about 9 Fr or less).

The delivery device 1706 may be used to deliver a “bulky knot” type tissue anchor (see, e.g., FIGS. 11 and 12). For example, the delivery device 1706 may be utilized to deliver a first tissue anchoring element 1702a on a distal side of a valve leaflet and/or a second anchoring element 1702b on a proximal side of a valve leaflet. In some instances, an elongate pusher may be movably disposed within a lumen of the catheter and/or elongate tube and/or coupled to a pusher hub that may be movably disposed within a handle and releasably coupled to the plunger. The delivery device 1706 carrying one or more pre-formed tissue anchoring element sutureforms 1702 can be movably disposed within a lumen of the pusher and coupled to a needle hub that is also coupled to the plunger. The plunger can be used to actuate or move the needle and the pusher during deployment of a distal anchoring element 1702 (e.g., the first anchoring element 1702a) and is movably disposed at least partially within the handle. For example, the handle may define a lumen in which the plunger can be moved. During operation, the pusher may also move within the lumen of the handle. A plunger lock can be used to prevent the plunger from moving within the handle during storage and prior to performing a procedure to deploy a tissue anchor.

The delivery device 1706 may have one or more pre-formed knot anchors 1702 disposed about a distal portion thereof while maintained in the catheter and/or elongate tube. For example, a pre-formed knot anchor 1702 may be formed of one or more sutures configured in a coiled sutureform having a plurality of winds/turns around the delivery device 1706 over a portion of the delivery device 1706 that is associated with a longitudinal slot in the delivery device 1706 that runs from the distal end thereof. Although the term “sutureform” is used herein, it should be understood that such components/forms may comprise suture, wire, or any other elongate material wrapped or formed in a desired configuration. The coiled sutureform can be provided or shipped disposed around the needle. In some examples, two suture tails 1703 extend from each coiled sutureform. The suture tails 1703 may extend through a passageway of the plunger and may exit the plunger at a proximal end portion thereof. The coiled sutureform may advantageously be configured to be formed into a suture-type tissue anchoring element (also referred to herein as a “bulky knot”) in connection with an anchor-deployment procedure, as described in more detail below. The coiled sutureform can be configurable to a knot/deployed configuration by approximating opposite ends of the coiled portion thereof towards each other to form one or more loops.

Advancement of the delivery device 1706 may be performed in conjunction with echo imaging, direct visualization (e.g., direct transblood visualization), and/or any other suitable remote visualization technique/modality. With respect to cardiac procedures, for example, the delivery system 1700 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue).

In some instances, the delivery device 1706 may be configured to penetrate (e.g., with the pointed tip 1707) a first leaflet of a heart valve (e.g., the mitral valve) within a ventricle (e.g., the left ventricle) and/or a second leaflet of the heart valve. The delivery device 1706 may be configured to deliver the first anchoring element 1702a at a distal side of the second leaflet and/or deliver the second anchoring element 1702b at a proximal side of the first leaflet. The first tail portion 1703a and/or the second tail portion 1703b may be configured to form a tethering suture tethered between the first anchoring element 1702a and the second anchoring element 1702b. The second anchoring element 1702b may be at least partially composed of a third tail portion 1703c and/or a fourth tail portion 1703d.

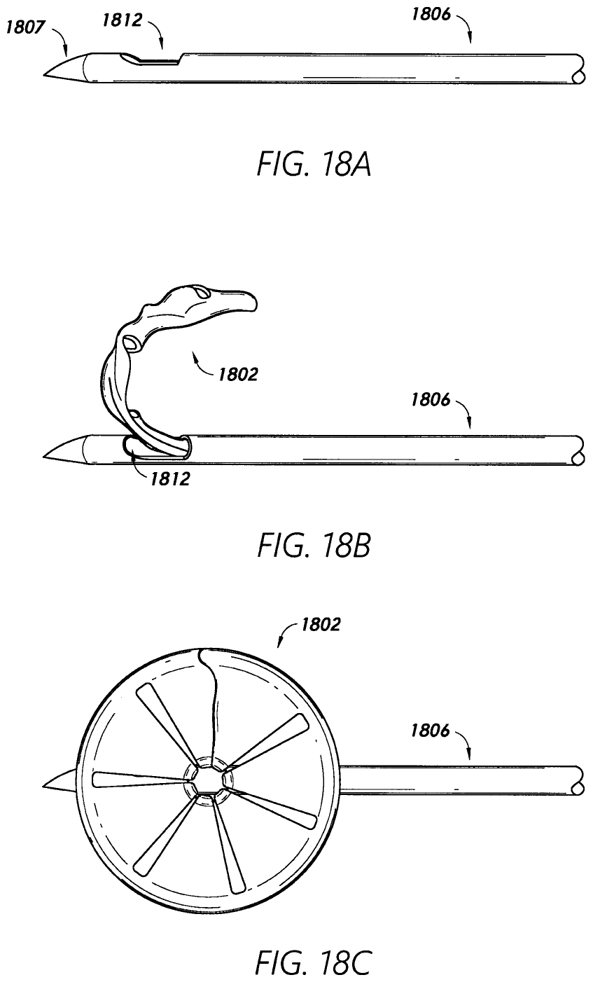

FIGS. 18A-18C illustrate another delivery system for delivering one or more anchoring elements 1802 to a valve leaflet in accordance with one or more examples. In some instances, a needle and/or other delivery device 1806 may have an at least partially hollow interior lumen configured to receive one or more anchoring elements 1802. Moreover, the delivery device may comprise one or more apertures 1812 to the interior lumen for delivery of the one or more anchoring elements 1802. One or more anchoring elements (e.g., sawtooth anchors) may be passed through the interior lumen of the delivery device 1806 and pushed through the aperture 1812 of the delivery device 1806 after the delivery device 1806 passes through one or more leaflets and/or arrives at a desired position at or near a valve leaflet. As shown in FIG. 18B, an anchoring element 1802 may be in an at least partially compressed form when passed through the delivery device 1806. In some instances, one or more anchoring elements 1802 may be composed of an at least partially flexible and/or elastic material to allow the anchoring element 1802 to be compressed while within the delivery device 1806. Moreover, an anchoring element 1802 may be configured to expand when pushed out of the delivery device 1806. One or more anchoring elements 1802 may be at least partially composed of a shape-memory material (e.g., Nitinol) such that when the anchoring element 1802 is removed from the delivery device 1806, the anchoring element 1802 assumes a predefined shape. For example, the anchoring element 1802 may comprise a shaping device formed of a shape-memory material (e.g., Nitinol) which may be configured to form the sawtooth anchoring element 1802 shown in FIG. 18C when removed from the delivery device 1806. A pusher and/or similar device may be utilized to advance the anchoring element 1802 out of the delivery device 1806.

In some instances, the delivery device 1806 may be configured to penetrate (e.g., with the pointed tip 1807) a first leaflet of a heart valve (e.g., the mitral valve) within a ventricle (e.g., the left ventricle) and/or a second leaflet of the heart valve. The delivery device 1806 may be configured to deliver the anchoring element 1802 at a distal side of the second leaflet and/or deliver a second anchoring element (e.g., a disc anchoring element and/or sawtooth anchoring element) at a proximal side of the first leaflet. One or more sutures may be configured to form a tethering suture tethered between the anchoring element 1802 and the second anchoring element.

### Valve Repair Processes

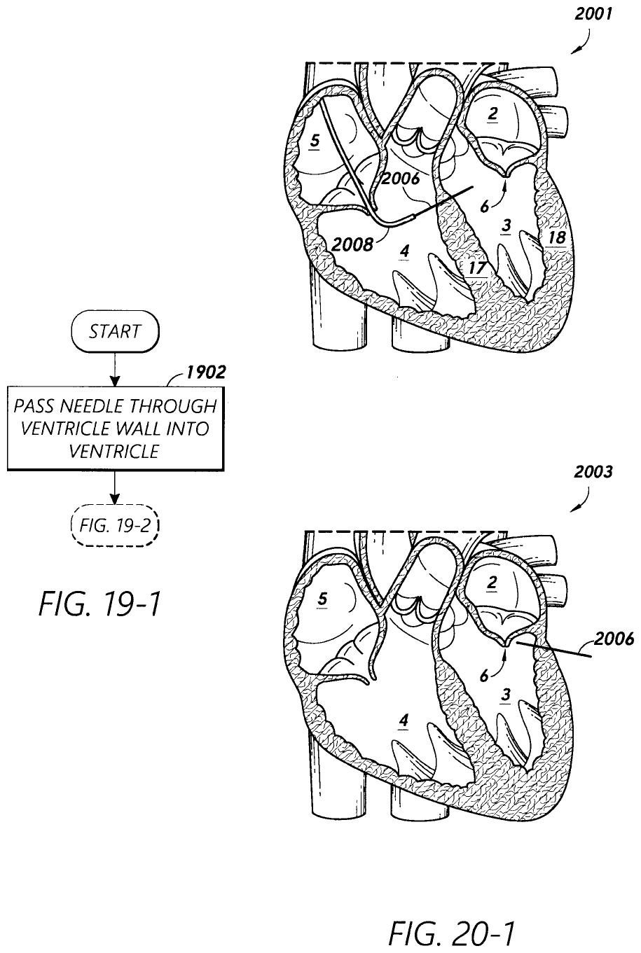

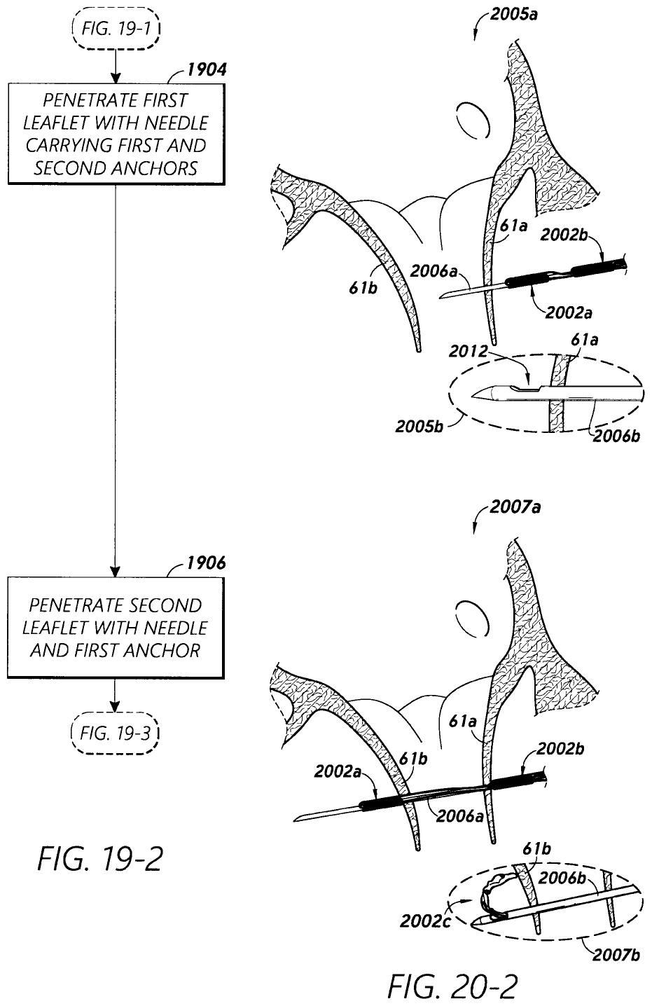

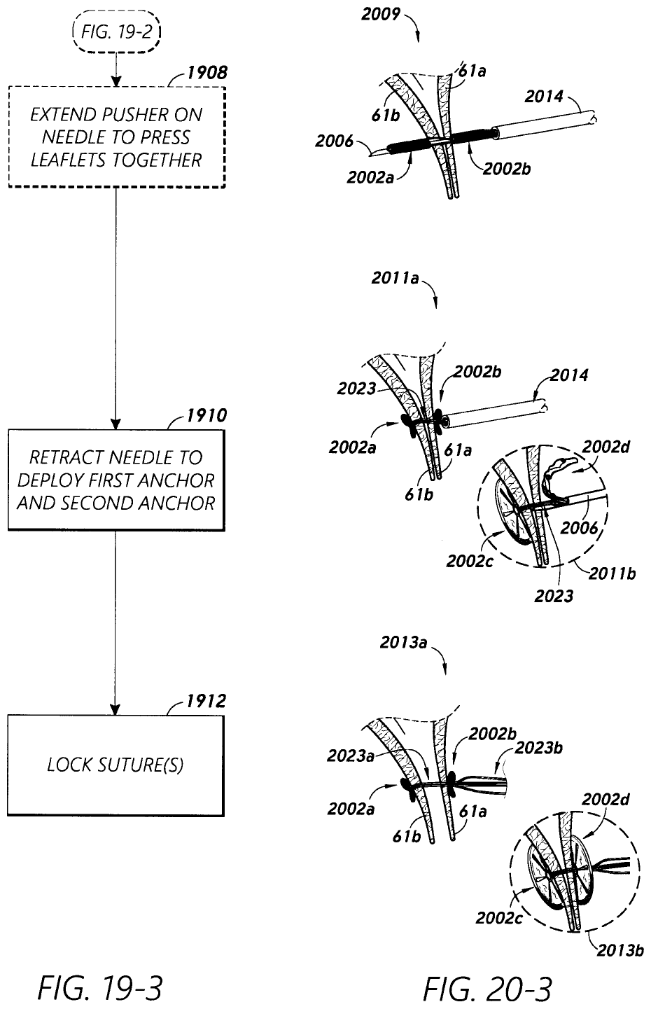

FIG. 19 (19-1, 19-2, and 19-3) provides a flow diagram representing a process for repairing a valve of the heart according to one or more instances disclosed herein. While some steps of the process may be directed to the left ventricle, such steps may also be applied to the right ventricle. FIG. 20 (18-1, 20-2, and 20-3) shows examples of various stages of the process for repairing a heart valve shown in FIG. 19.

At step 1902, the process involves inserting/passing a delivery device 2006 (e.g., a needle) through a ventricle wall and into a target ventricle. The ventricle wall may be a septum 17, as shown in image 2001 of FIG. 20-1 and/or a posterior wall 20, as shown in image 2003 of FIG. 20-1. The delivery device 2006 and/or one or more coaptation devices may be inserted into the heart via a catheter 2008 and/or other percutaneous procedure. For example, the delivery device 2006 may be delivered using a transfemoral, transendocardial, transcoronary, transseptal, and/or transapical procedure, or other approach. In optional instances, the delivery device and/or one or more coaptation devices may be introduced into the desired location during an open-chest surgical procedure, or using other surgical or non-surgical techniques known in the art. The one or more coaptation devices may include one or more cords (e.g., sutures) and/or anchoring elements (e.g., sutureforms, knots, sawtooth anchoring elements).

The delivery device 2006 may be passed through the ventricle wall (e.g., the septum 17) at a point that causes the delivery device 2006 to enter the target ventricle (e.g., the left ventricle 3) at or near a target valve (e.g., the mitral valve 6). For example, the delivery device 2006 may be delivered via the right atrium 5, pulmonary valve 9, and/or other area and into the right ventricle 4 and may penetrate and/or pass through the septum 17 at an upper portion of the septum 17 (e.g., near the pulmonary valve 9). When the delivery device 2006 and/or catheter 2008 exit the septum 17, the delivery device 2006 and/or catheter 2008 may be situated near the mitral valve 6. Similarly, the delivery device 2006 and/or catheter 2008 may be passed through a portion of the posterior wall 20 that causes the delivery device 2006 and/or catheter 2008 to exit the posterior wall 20 at or near the mitral valve 6. In this way, the delivery process may provide efficient and/or simplified access to the target valve (e.g., the mitral valve 6). In some instances, the delivery device 2006 may be configured to enter the right ventricle 4 from either a jugular or femora access point. The delivery device 2006 may be steered toward the interventricular septum 17 with a trajectory toward the A2 coaptation zone of the anterior mitral leaflet.

In some instances, the delivery device 2006 may be inserted into the right ventricle 4 (e.g., through the pulmonary valve or tricuspid valve) to deploy one or more coaptation devices for repairing the right ventricle 4 or may be passed through the septum 17 into the left ventricle 3. Alternatively, the delivery device 2006 may be inserted into the left ventricle 3 (e.g., through the aortic valve or mitral valve) to deploy one or more coaptation devices for repairing the left ventricle 3 or may be passed through the septum 17 into the right ventricle 4. For a transapical procedure, the delivery device 2006 may be inserted through the apex via a catheter 2008. In optional instances, the delivery device may be delivered to a location outside of the heart for purposes other than valve repair.

In some instances, the delivery device 2006 may be fed through a catheter 2008 (e.g., a transfemoral catheter) that may be inserted into the left ventricle 3 or right ventricle 4. Needles and/or other delivery devices 2006 may be passed through the catheter 2006 to penetrate the septum 17 and/or other ventricle walls (e.g., one or more posterior walls 20). For example, a transseptal needle may be introduced to pass through the septum 17 from the right ventricle 4 to the left ventricle 3. The catheter may be sized to accommodate the various elements of the coaptation device.

The coaptation device may be positioned to cause repairing of a valve while avoiding damage to the papillary muscles, chordae tendineae, and/or other heart anatomy. For example, the various cords and/or anchoring elements may be positioned to avoid contacting the papillary muscles during delivery and after delivery of the coaptation device.

At step 1904, the process involves penetrating a first valve leaflet 61a with the delivery device 2006. Image 2005a depicts a first example in which one or more anchoring elements 2002 are wrapped around an exterior of a first delivery device 2006a. Image 2005b depicts an additional and/or alternative example in which one or more anchoring elements 2002 may be delivered via an aperture 2012 and/or internal lumen of a second delivery device 2006b. The delivery device 2006 may be configured to carry and/or deliver one or more anchoring elements 2002 (e.g., a first suture anchoring element 2002a and/or a second suture anchoring element 2002b). In some instances, the one or more anchoring elements may comprise one or more sutureforms configured to be formed into bulky knots. The first suture anchoring element 2002a and/or second suture anchoring element 2002b may be wrapped around an outer surface of the delivery device 2006.

The delivery device 2006 may be configured to penetrate the first valve leaflet 61a at any suitable point of the first valve leaflet 61a. In some instances, the delivery device 2006 may be configured to penetrate the first valve leaflet 61a at or near an edge portion of the first valve leaflet 61a and/or at or near a central region of the first valve leaflet 61a (see, e.g., FIG. 4C).

Image 2005b illustrates a cutout view of an alternative second delivery device 2006b comprising at least one aperture 2012 configured to provide an opening/aperture 2012 to an internal lumen of the second delivery device 2006b. The second delivery device 2006b may be utilized in place of the first delivery device 2006a to deliver one or more anchoring elements 2002 via the internal lumen of the second delivery device 2006b. In some instances, a delivery device 2006 configured to carry one or more anchoring elements 2002 at an outer surface of the delivery device (e.g., the first delivery device 2006a) may also have at least one aperture 2012 and/or an internal lumen for delivering anchoring elements via the internal lumen.

At step 1906, the process involves penetrating a second valve leaflet 61b with the delivery device 2006 and/or a first suture anchoring element 2002a (see image 2007a) and/or a first sawtooth anchoring element 2002c (see image 2007b). The delivery device 2006 may have a sufficient length that the delivery device 2006 may be configured to extend a complete distance between the first leaflet 61a and the second leaflet 61b and/or extend beyond the second leaflet 61b. The delivery device 2006 (e.g., a first delivery device 2006a in image 2007a and/or a second delivery device 2006b in image 2007b) may be extended until a first suture anchoring element 2002a (see image 2007a) and/or an aperture 2012 of the delivery device 2006 is completely or at least partially extended beyond the second leaflet 61b. The delivery device 2006 may be configured to create a sufficiently large opening to allow the first suture anchoring element 2002a to pass through the first leaflet 61a and/or the second leaflet 61b. The first sawtooth anchoring element 2002c may be in a compressed form while within the delivery device 2006.

As shown in images 2007a and 2007b, the first suture anchoring element 2002a and/or first sawtooth anchoring element 2002c may be configured to be delivered at a distal side of the second leaflet 61b. In other words, the first suture anchoring element 2002a and/or first sawtooth anchoring element 2002c may be configured for delivery at a side of the second leaflet 61b that is distal to an entry point of the delivery device 2006 into the ventricle. For example, if the delivery device 2006 is delivered into the left ventricle 3 via the septum 17 (see, e.g., image 2001), the delivery device 2006 may first pass through an anterior leaflet of the mitral valve 6 (e.g., at the A1, A2, or A3 segment), then a posterior leaflet of the mitral valve 6 (e.g., at the P1, P2, or P3 segment), and the first suture anchoring element 2002a and/or first sawtooth anchoring element 2002c may be delivered to a distal side of the posterior leaflet (e.g., the side facing the posterior wall 20). If the delivery device 2006 is delivered into the left ventricle 3 via the posterior wall 20 (see, e.g., image 2003), the delivery device 2006 may first pass through a posterior leaflet of the mitral valve 6, then an anterior leaflet of the mitral valve 6, and the first suture anchoring element 2002a and/or first sawtooth anchoring element 2002c may be delivered to a distal side of the an anterior (e.g., the side facing the septum 17). To create coaptation at a middle portion of the mitral valve 6, it may be advantageous to penetrate the mitral valve 6 at the A2 and/or P2 segments.

In some instances, the process includes a step 1908 involving extending a pusher 2014 and/or similar device over the delivery device 2006 to press the first suture anchoring element 2002a and second suture anchoring element 2002b together and/or to press the first leaflet 61a and/or second leaflet 61b together, as shown in image 2009 of FIG. 20-3. In examples in which a sawtooth anchoring element is utilized, it may not be necessary to extend a pusher 2014 to press the anchoring elements 2002 together. However, a pusher 2014 and/or similar device may be extended through an internal lumen of the delivery device 2006 to push the first sawtooth anchoring element 2002c out of the aperture 2012.

The pusher 2014 may comprise an internal lumen configured to fit the delivery device 2006 within the internal lumen of the pusher 2014 and/or may be extended along the delivery device 2006 until the first leaflet 61a and second leaflet 61b are suitably approximated. The internal lumen of the pusher 2014 may be large enough to fit the delivery device 2006 but may not be large enough to fit around the second suture anchoring element 2002b while the second suture anchoring element 2002b is wrapped around the delivery device 2006. Thus, when the pusher 2014 reaches the second suture anchoring element 2002b, the pusher 2014 may be configured to apply pushing force to the second suture anchoring element 2002b. In some instances, the first suture anchoring element 2002a and/or second suture anchoring element 2002b may be slidably attached to the delivery device 2006. For example, when pushing force from the pusher 2014 is applied to the second suture anchoring element 2002b, the second suture anchoring element 2002b may be configured to slide along the delivery device 2006 toward the first suture anchoring element 2002a to reduce the distance between the first suture anchoring element 2002a and the second suture anchoring element 2002b.

In some instances, an opening created in the first leaflet 61a by the delivery device 2006 may be sufficiently small to provide resistance to the second suture anchoring element 2002b when the second suture anchoring element 2002b is pressed against the first leaflet 61a. For example, the second suture anchoring element 2002b, at least while wrapped about the delivery device 2006, may have a coil diameter that is greater than a diameter of the opening in the first valve created by the delivery device 2006. In some instances, the second suture anchoring element 2002b may have a greater coil diameter than the first suture anchoring element 2002a. However, in some instances the opening in the first leaflet 61a created by the delivery device 2006 may be sufficiently large to fit the second suture anchoring element 2002b but may be sufficiently small to present at least a minimal amount of resistance to the second suture anchoring element 2002b to prevent the second suture anchoring element 2002b from entering the first leaflet 61a.

As the pusher 2014 presses against the second suture anchoring element 2002b, the first leaflet 61a may present sufficient resistance that the first leaflet 61a may be pushed towards the second leaflet 61b. The first leaflet 61a may be moved toward the second leaflet 61b until the first leaflet 61a contacts the second leaflet 61b. When the first leaflet 61a and second leaflet 61b are in contact, the second leaflet 61b may provide resistance to the first leaflet 61a and/or may provide additional resistance to prevent the second suture anchoring element 2002b from passing through the opening in the first leaflet 61a. Moreover, the first leaflet 61a pressing against the second leaflet 61b may additionally or alternatively provide resistance to prevent the first suture anchoring element 2002a from passing through the second leaflet 61b.

At step 1910, the process involves at least partially retracting the delivery device 2006. As shown in image 2011a of FIG. 20-3, the first suture anchoring element 2002a and/or the second suture anchoring element 2002b may be deployed and/or may remain at the leaflets 61 following retraction of the delivery device 2006. Moreover, the first suture anchoring element 2002a and/or second suture anchoring element 2002b may be configured to form the bulky knot form shown in image 2011a when removed from the delivery device 2006. In this way, the first suture anchoring element 2002a and/or the second suture anchoring element 2002b may create a secure attachment to the leaflets as the bulky knot form may be difficult to fit through openings made in the leaflets 61.

In some instances, the pusher 2014 may remain in contact with the second suture anchoring element 2002b while the delivery device 2006 is retracted. In this way, the first suture anchoring element 2002a and/or the second suture anchoring element 2002b may be configured to slide off of the delivery device 2006 in response to pressure applied by the pusher 2014.

One or more tail portions of the first anchoring element 2002 and/or second anchoring element 2002b may be cinched to cause the anchoring elements 2002 to form the bulky knot forms shown in image 2011a. In some instances, the first anchoring element 2002a and second anchoring element 2002b may be cinched sequentially or simultaneously. For example, when the delivery device is retracted, the first anchoring element 2002a may be removed from the delivery device before the second anchoring element 2002b is removed from the delivery device. Once the first anchoring element 2002a is removed from the delivery device, one or more tail portions of the first anchoring element 2002a may be pulled and/or otherwise cinched to cause the first anchoring element 2002a to form a bulky knot while the second anchoring element 2002b is still wrapped around the delivery device. After the first anchoring element 2002a is formed into a bulky knot, the second anchoring element 2002b may be removed (e.g., via pushing force from the pusher 2014) and/or formed into a bulky knot. In some instances, the first anchoring element 2002a and/or second anchoring element 2002b may naturally form a bulky knot when removed from the delivery device.

In the example shown in image 2011b of FIG. 20-3, a first sawtooth anchoring element 2002c may remain deployed at the second leaflet 61b when the delivery device 2006 is retracted out of the second leaflet 61b. A second sawtooth anchoring element 2002d may be deployed out of the aperture 2012 of the second delivery device 2006b when the aperture 2012 is at a proximal side of the first leaflet 61a. In this way, the second sawtooth anchoring element 2002d may be configured to contact and/or attach to the proximal side of the first leaflet 61a. In some instances, the second sawtooth anchoring element 2002d may be replaced by any suitable anchor, for example a disc anchoring element (see, e.g., FIG. 14). One or more sutures and/or suture tails 2023 may be configured to tether the first suture anchoring element 2002a to the second suture anchoring element 2002b and/or to tether the first sawtooth anchoring element 2002c to the second sawtooth anchoring element 2002d.

At step 1912, the process involves locking one or more sutures and/or suture tails 2023 to maintain a distance between the first suture anchoring element 2002a and the second suture anchoring element 2002b (see image 2013a of FIG. 20-3), between the first sawtooth anchoring element 2002c and the second sawtooth anchoring element 2002d (see image 2013b of FIG. 20-3), and/or between the first leaflet 61a and the second leaflet 61b. A first portion 2023a of the suture tails may form a tether between the anchoring elements 2002 while a second portion 2023b of the suture tails may form one or more knots and/or may attach to a locking mechanism at the proximal side of the first leaflet 61a.

As shown in images 2013a and 2013b, the second suture anchoring element 2002b and/or second sawtooth anchoring element 2002d (or other type of anchoring element) may be configured to be delivered at a proximal side of the first leaflet 61a. For example, the second suture anchoring element 2002b and/or second sawtooth anchoring element 2002d may be delivered at a first penetration point of the delivery device 2006 at the first leaflet 61a.

The process shown in FIGS. 19 and 20 and/or other processes, devices, and systems disclosed herein may advantageously provide mechanisms for implementing valvular repairing using a fully transcatheter procedure on a beating heart. Furthermore, in certain instances, the various devices may be designed to be retrievable.

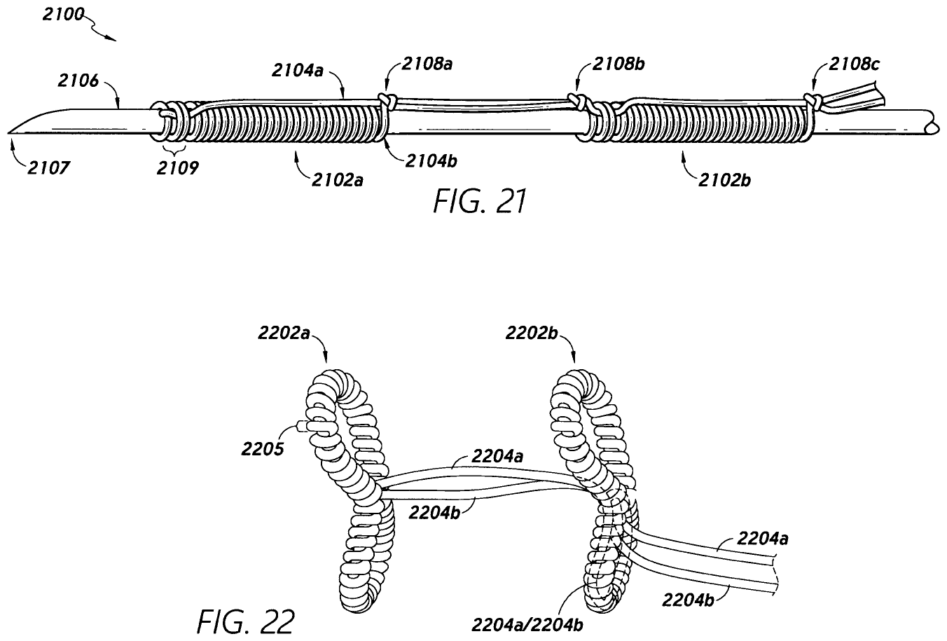

FIG. 21 shows a coaptation device comprising a first anchoring element 2102a and a second anchoring element 2102b in accordance with one or more examples. In some examples, one or more anchoring elements 2102 may comprise one or more sutures formed into a sutureform. An anchoring element 2102 formed at least partially of a suture may also be referred to herein as a “suture anchor” or “suture anchoring element.” A sutureform may comprise an at least partially coiled suture comprising one or more coils 2109. The sutureform may be configured to be formed into a bulky knot, as shown in FIG. 22. One or more tail portions 2104 (e.g., ends) of an anchoring element 2102 may be configured to extend from and/or may be an extension (e.g., a free end) of the sutureform to attach to, extend into, interconnect with, and/or interlock with another sutureform and/or other type of anchoring element 2102. Accordingly, one or more tail portions 2104 may form tethering sutures between multiple anchoring elements 2102.

In some examples, a suture forming one or more anchoring elements 2102 may also serve as a tethering suture between multiple anchoring elements 2102. For example, a first (e.g., distal) anchoring element 2102a may be at least partially formed of a suture having two tail portions 2104 (e.g., a first tail portion 2104a and a second tail portion 2104b) which may be configured to extend into and/or secure to a second (e.g., proximal) anchoring element 2102b. In some instances, a suture forming the first anchoring element 2102a may also form the second anchoring element 2102b. For example, the first tail portion 2104a and/or the second tail portion 2104b extending from the first anchoring element 2102a may be configured to coil around a delivery device 2106 (e.g., a needle) to form the second anchoring element 2102b.

One or more tail portions 2104 extending from the first anchoring element 2102a may be configured to fit through and/or pass under one or more coils 2109 of the second anchoring element 2102b such that at least a portion of the second anchoring element 2102b may be wrapped around one or the one or more tail portions 2104 of the first anchoring element 2102a. For example, a second tail portion 2104b may be coiled around the delivery device 2106 while a first tail portion 2104a may pass through at least a portion of the coils formed by the second tail portion 2104b.

The first anchoring element 2102a and/or second anchoring element 2102b may be coiled to form an internal lumen through the one or more coils/windings 2109 of the anchoring element 2102 such that the delivery device 2106 may pass at least partially through the first anchoring element 2102a and/or the second anchoring element 2102b. In some examples, the one or more tail portions 2104 extending from the first anchoring element 2102a (e.g., the first tail portion 2104a and/or second tail portion 2104b) may at least partially form the second anchoring element 2102b. In other words, the first anchoring element 2102a and the second anchoring element 2102b may be formed from a common set of one or more sutures (e.g., a single suture). In such cases, the two tail portions 2104 extending from the first anchoring element 2102a may also extend from the second anchoring element 2102b.

The delivery device 2106 (e.g., needle) may comprise a pointed tip 2107 configured to penetrate and/or pass through one or more valve leaflets. The needle 2106 may be configured to carry the first (e.g., distal) anchoring element 2102a and/or the second (e.g., proximal) anchoring element 2102b. The one or more anchoring elements 2102 may be configured to be delivered simultaneously or sequentially. In some examples, the first anchoring element 2102a and/or the second anchoring element 2102b may be configured to form a bulky knot when removed from the delivery device 2106. In some cases, a pusher or similar device may be configured to push one or more of the anchoring elements 2102 off of the needle 2106.

The first suture tail 2104a and/or second suture tail 2104b may be configured to extend out of the second anchoring element 2102b and/or out of the ventricle and may be accessible to a surgeon. When the first suture tail 2104a and/or second suture tail 2104b is/are pulled, the coils/windings of the second anchoring element 2102b may be configured to circularize to form a bulky knot (e.g., a figure-8 knot; see, e.g., FIG. 22).

In some instances, the first suture tail 2104a and/or second suture tail 2104b may be configured to form one or more knots 2108 at end portions of the anchoring elements 2102. For example, the first suture tail 2104a and/or second suture tail 2104b may be knotted together to form a first knot 2108a at an end portion of the first anchoring element 2102a (e.g., between the first anchoring element 2102a and the second anchoring element 2102b). The first suture tail 2104a and/or second suture tail 2104b may additionally or alternatively be knotted together to form a second knot 2108b at a proximal end portion of the second anchoring element 2102b (e.g., between the first anchoring element 2102a and the second anchoring element 2102b) and/or a third knot 2108c at a distal end portion of the second anchoring element 2102b. The knots 2108 may be configured to prevent the first anchoring element 2102a and/or the second anchoring element 2102b from unraveling, loosening, and/or separating from the delivery device 2106 and/or from each other. In some instances, a knot 2108 may be formed from a single suture tail 2104 and/or from two suture tails 2104 being knotted together.

The delivery device 2106 may be configured to be inserted into the heart via a catheter and/or an elongate tube forming at least one internal working lumen. Implementation of a valve-repair procedure utilizing the delivery system 2100 can be performed in conjunction with certain imaging technology designed to provide visibility of the delivery device 2106, catheter, and/or other components of the delivery system 2100 according to a certain imaging modality, such as echo imaging. Generally, when performing a valve-repair procedure utilizing the tissue anchoring element delivery system 2100, the operating physician may advantageously work in concert with an imaging technician, who may coordinate with the physician to facilitate successful execution of the valve-repair procedure.

In addition to the catheter and/or elongate tube, the delivery system 2100 may include a plunger feature, which may be used and/or actuated to manually deploy the delivery device 2106 and/or to remove the one or more anchoring elements 2102 from the delivery device 2106. The lumen of the catheter and/or elongate tube may be configured to house the delivery device 2106 wrapped at least in part with one or more pre-formed knot sutureform anchors 2102. In some examples, the catheter and/or elongate tube presents a relatively low profile. For example, a catheter may have a diameter of approximately 3 mm or less (e.g., about 9 Fr or less).

The delivery device 2106 may be used to deliver a “bulky knot” type tissue anchor (see, e.g., FIG. 22). For example, the delivery device 2106 may be utilized to deliver a first tissue anchoring element 2102a on a distal side of a valve leaflet and/or a second anchoring element 2102b on a proximal side of a valve leaflet. In some instances, an elongate pusher may be movably disposed within a lumen of the catheter and/or elongate tube and/or coupled to a pusher hub that may be movably disposed within a handle and releasably coupled to the plunger. The delivery device 2106 carrying one or more pre-formed tissue anchoring element sutureforms 2102 can be movably disposed within a lumen of the pusher and coupled to a needle hub that is also coupled to the plunger. The plunger can be used to actuate or move the needle and the pusher during deployment of a distal anchoring element 2102 (e.g., the first anchoring element 2102a) and is movably disposed at least partially within the handle. For example, the handle may define a lumen in which the plunger can be moved. During operation, the pusher may also move within the lumen of the handle. A plunger lock can be used to prevent the plunger from moving within the handle during storage and prior to performing a procedure to deploy a tissue anchor.

The delivery device 2106 may have one or more pre-formed anchors 2102 disposed about a distal portion thereof while maintained in the catheter and/or elongate tube. For example, a pre-formed anchor 2102 may be formed of one or more sutures configured in a coiled sutureform having a plurality of winds/turns around the delivery device 2106 over a portion of the delivery device 2106 that is associated with a longitudinal slot in the delivery device 2106 that runs from the distal end thereof. Although the term “sutureform” is used herein, it should be understood that such components/forms may comprise suture, wire, or any other elongate material wrapped or formed in a desired configuration. The coiled sutureform can be provided or shipped disposed around the needle. In some examples, two suture tails 2104 extend from each coiled sutureform. The suture tails 2104 may extend through a passageway of the plunger and may exit the plunger at a proximal end portion thereof. The coiled sutureform may advantageously be configured to be formed into a suture-type tissue anchoring element (also referred to herein as a “bulky knot”) in connection with an anchor-deployment procedure, as described in more detail below. The coiled sutureform can be configurable to a knot/deployed configuration by approximating opposite ends of the coiled portion thereof towards each other to form one or more loops.

Advancement of the delivery device 2106 may be performed in conjunction with echo imaging, direct visualization (e.g., direct transblood visualization), and/or any other suitable remote visualization technique/modality. With respect to cardiac procedures, for example, the delivery device 2106 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue).

In some instances, the delivery device 2106 may be configured to penetrate (e.g., with the pointed tip 2107) a first leaflet of a heart valve (e.g., the mitral valve) within a ventricle (e.g., the left ventricle) and/or a second leaflet of the heart valve. The delivery device 2106 may be configured to deliver the first anchoring element 2102a at a distal side of the second leaflet and/or deliver the second anchoring element 2102b at a proximal side of the first leaflet. The first tail portion 2104a and/or the second tail portion 2104b may be configured to form a tethering suture tethered between the first anchoring element 2102a and the second anchoring element 2102b.

FIG. 22 illustrates a coaptation device comprising two anchoring elements forming bulky knot anchors (i.e., bulky knots). In some examples, each of the suture anchoring elements in FIG. 21 may be configured to form a bulky knot anchor. For example, a sutureform may be configured to form a bulky knot anchor when the sutureform is removed from a from a delivery device (e.g., a needle). The bulky knot anchors formed by the sutureforms may create one or more openings and/or loop portions. For example, a bulky knot anchor may form a figure-8 pattern comprising two interlocking loop portions, as shown in FIG. 22. One or more tail portions 2204 of the first anchoring element 2202a may be configured to form the second anchoring element 2202b and/or may be configured to extend through the one or more openings formed in the second anchoring element 2202b and/or may pass through one or more coils 2205 of the second anchoring element 2202b. In some instances, one or more tail portions 2204 from the first anchoring element 2202a (e.g., the first tail portion 2204a and/or second tail portion 2204b) may be configured to form a tether between the first anchoring element 2202a and the second anchoring element 2202b. Accordingly, when the one or more tail portions 2204 of the first anchoring element 2202a are cinched, the one or more tail portions 2204 may be configured to reduce the distance between the first anchoring element 2202a and the second anchoring element 2202b.

In some instances, one or more fasteners and/or suture locking devices may be additionally or alternatively used to lock the tail portions 2204 and/or anchoring elements 2202 in place. For example, the first tail portion 2204a and/or second tail portion 2204b may be configured to form one or more knots at end portions of the first anchoring element 2202a and/or second anchoring element 2202b to hold the tail portions 2204 and/or anchoring elements 2202 in place (see, e.g., the knots 2108 in FIG. 21). In some instances, knots formed in the suture(s) forming the first anchoring element 2202a and/or second anchoring element 2202b may be configured to lock the first anchoring element 2202a and the second anchoring element 2202b relative to each other. The first tail portion 2204a and/or the second tail portion 2204b may represent tethering sutures and may be configured to tether the first anchoring element 2202a to the second anchoring element 2202b.

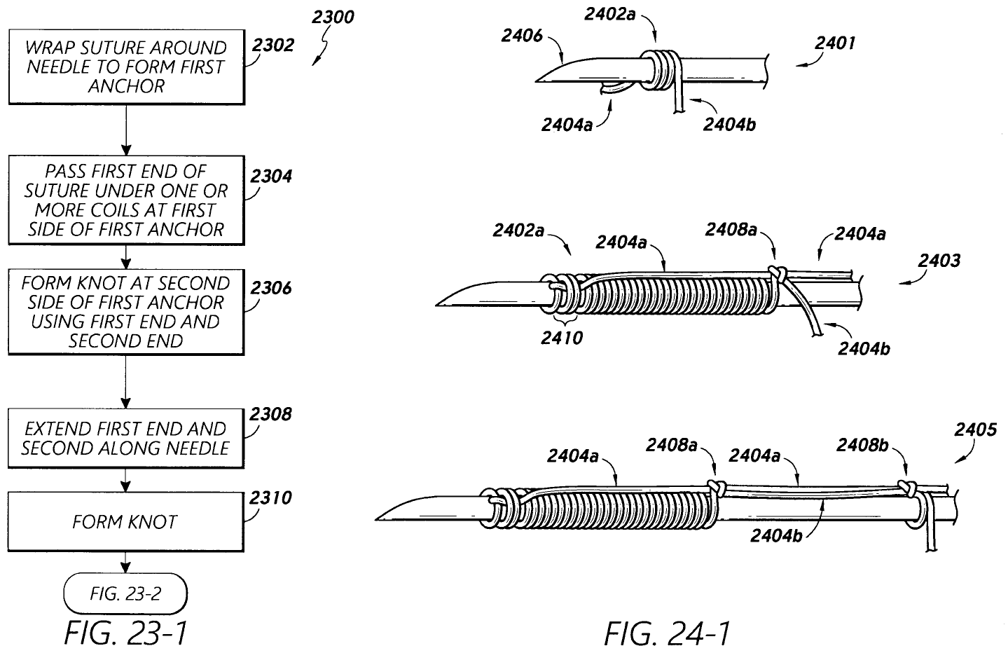

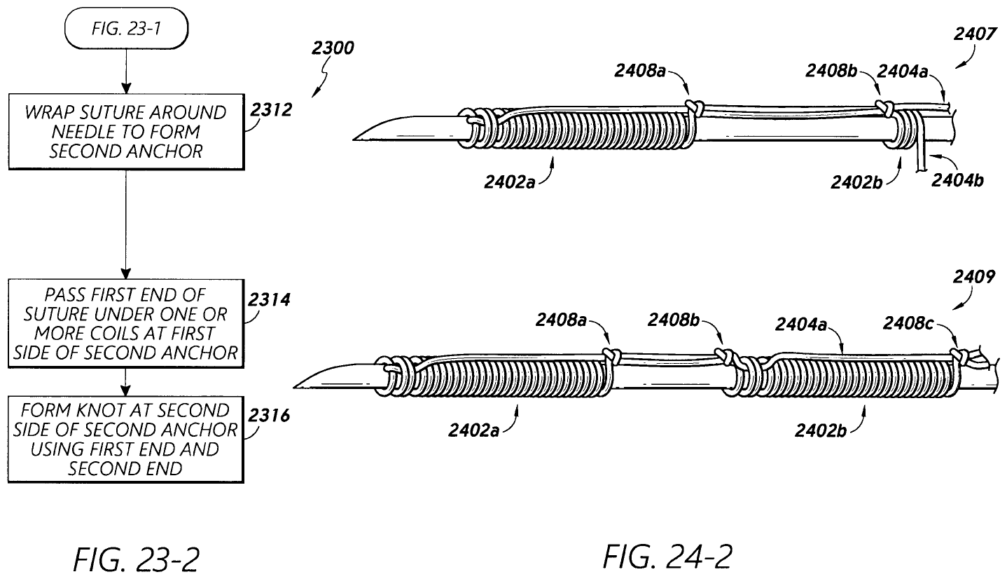

FIG. 23 (23-1 and 23-2) provides a flow diagram representing a process 2300 for wrapping a suture onto a delivery device according to one or more instances disclosed herein. FIG. 24 (24-1 and 24-2) shows examples of various stages of the process 2300 for wrapping the suture onto the delivery device shown in FIG. 23.

At step 2302, the process 2300 involves wrapping a suture (comprising a first suture tail portion 2404a and a second suture tail portion 2404b) around a delivery device 2406 (e.g., a needle) to form a first anchoring element 2402a, as shown in image 2401 of FIG. 24. In some instances, the suture may be slid through a needle skive and/or similar device. For example, the suture may be slid through a needle skive under both needles of the needle skive. The suture slack (e.g., the first tail portion 2404a and/or the second tail portion 2404b) may be evenly distributed on either side of the needle skive. Only one side of the suture (e.g., the second tail portion 2404b) may be wound (e.g., around the delivery device 2406).

One suture tail portion (e.g., the first tail portion 2404a) may be held taut while a second suture tail portion (e.g., the second tail portion 2404b) may be wound over the delivery device 2406. In some instances, a winder carriage may be rotated (e.g., in a clockwise direction) to wind the second suture tail portion 2404b onto the delivery device 2406. During the winding process, the coils may be tightened to keep the coils compact and uniform. In some instances, the second tail portion 2404b may be wound approximately fifteen times around the delivery device 2406 to form approximately fifteen coils.

At step 2304, the process 2300 involves passing the first tail portion 2404a under one or more coils 2410 of the first anchoring element 2402a, as shown in image 2403 of FIG. 24. The first tail portion 2404a may be passed under any number of coils 2410 (e.g., two) and/or under coils 2410 at any portion of the first anchoring element 2402a (e.g., at a proximal side of the first anchoring element 2402a, as shown in image 2403 of FIG. 24). By passing the first tail portion 2404a under one or more coils 2410 of the first anchoring element 2402a, the first tail portion 2404a may advantageously be securely held in place to prevent unraveling and/or loosening of the first anchoring element 2402a.

At step 2306, the process 2300 involves forming a first knot 2408a at a distal side of the first anchoring element 2402a, as shown in image 2403 of FIG. 24. The first knot 2408a may be formed by knotting the first tail portion 2404a and the second tail portion 2404b together and/or by knotting the first tail portion 2404a and/or the second tail portion 2404b to themselves. The first knot 2408a may be configured to advantageously hold the first anchoring element 2402a in place to prevent sliding, loosening, and/or unraveling of the first anchoring element 2402a.

At step 2308, the process 2300 involves extending the first tail portion 2404a and/or the second tail portion 2404b along the delivery device 2406 to create a gap between the first anchoring element 2402a and a second anchoring element to be formed later, as shown in image 2405 of FIG. 24.

At step 2310, the process 2300 involves forming a second knot 2408b at the delivery device 2406, as shown in image 2405 of FIG. 24. The first tail portion 2404a and/or the second tail portion 2404b may be configured to form a second knot 2408b around delivery device 2406. There may be any size of gap between the first knot 2408a and the second knot 2408b. For example, the gap between the first knot 2408a and the second knot 2408b may be approximately equivalent to a length of the first anchoring element 2402a when the first anchoring element 2402a is wrapped in a sutureform around the delivery device 2406, as shown in image 2405 of FIG. 24.

At step 2312, the process 2300 involves wrapping the suture around the delivery device 2406 to form a second anchoring element 2402b, as shown in image 2407 of FIG. 24. The suture may be wrapped on a side of the second knot 2408b that is distal from the first anchoring element 2402a. Accordingly, each successive coil forming the second anchoring element 2402b may be situated increasingly distant from the first anchoring element 2402a. In some instances, the first tail portion 2404a and/or the second tail portion 2404b may be inserted inside a needle skive prior to forming the second anchoring element 2402b.

At step 2314, the process 2300 involves passing the first suture tail 2404a under one or more coils formed by the second suture tail 2404b, as shown in image 2409 of FIG. 24. The first suture tail 2404a may be passed under any number of coils and/or may be passed under coils at any portion of the second anchoring element 2402b.

At step 2316, the process 2300 involves forming a third knot 2408c at the completion of the second anchoring element 2402b, as shown in image 2409 of FIG. 24. In some instances, the first suture tail 2404a and/or the second suture tail 2404b may be knotted together and/or to themselves to form the third knot 2408c. The second knot 2408b and/or third knot 2408c may advantageously prevent the second anchoring element 2402b from unraveling and/or loosening.

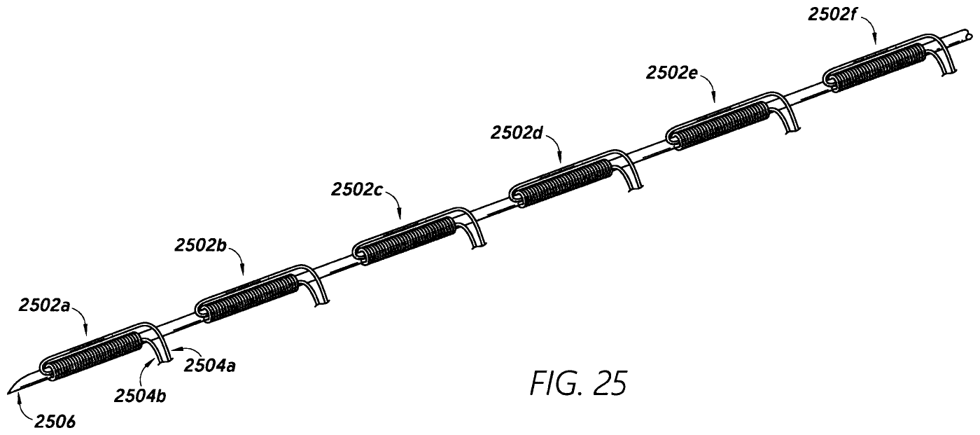

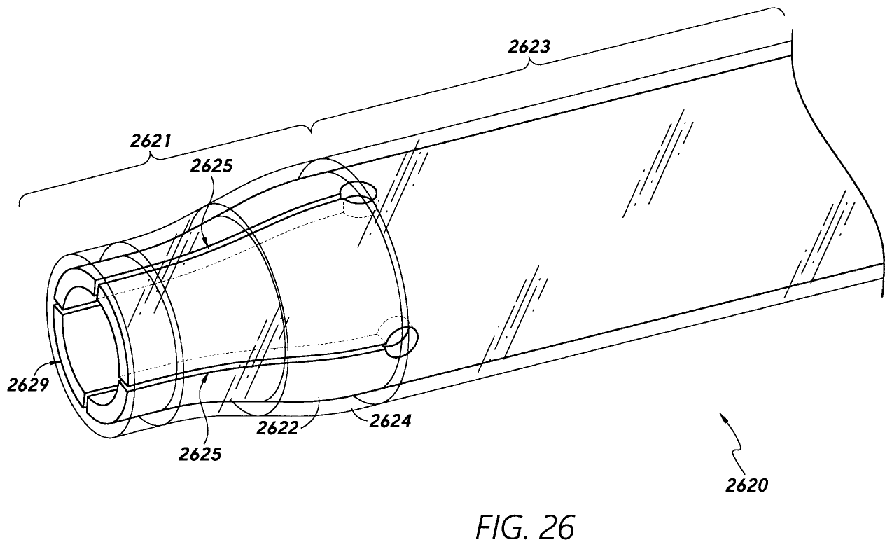

FIG. 25 illustrates an example delivery system 2500 for delivering one or more anchoring elements 2502 to a valve leaflet in accordance with some examples. In the example shown in FIG. 25, six sutureform anchoring elements 2502 (e.g., a first suture anchoring element 2502a, a second suture anchoring element 2502b a third suture anchoring element 2502c, a fourth suture anchoring element 2502d, a fifth suture anchoring element 2502e, and a sixth suture anchoring element 2502f) may be at least partially wrapped around a delivery device 2506 (e.g., a needle) comprising a pointed tip configured to penetrate and/or pass through one or more valve leaflets. The anchoring elements 2502 may be configured to be delivered simultaneously or sequentially. In some examples, the anchoring elements 2502 may be configured to form bulky knot anchors when removed from the needle 2506. In some cases, a pusher (see, e.g., FIG. 26) and/or similar device may be configured to receive one or more anchoring elements 2502 within a lumen of the pusher and/or to push one or more of the anchoring elements 2502 off of the needle 2506. In some examples, the various anchoring elements 2502 may not be interlocked and/or otherwise attached to each other. The anchoring elements 2502 may be configured to be delivered independently and/or sequentially.

Each of the anchoring elements 2502 may comprise two suture tails 2504 (e.g., a first suture tail 2504a and/or a second suture tail 2504b) configured to extend out of the ventricle to be accessible to a surgeon. When the suture tails 2504 are pulled, the coils/windings of the anchoring elements 2502 may be configured to circularize to form a bulky knot.

In some instances, the delivery device 2506 may be configured to be inserted into the heart via a catheter and/or an elongate tube forming at least one internal working lumen. Implementation of a valve-repair procedure utilizing the delivery system 2500 can be performed in conjunction with certain imaging technology designed to provide visibility of the delivery device 2506, catheter, and/or other components of the delivery system 2500 according to a certain imaging modality, such as echo imaging. Generally, when performing a valve-repair procedure utilizing the tissue anchoring element delivery system 2500, the operating physician may advantageously work in concert with an imaging technician, who may coordinate with the physician to facilitate successful execution of the valve-repair procedure.

In addition to the catheter and/or elongate tube, the delivery system 2500 may include a plunger feature, which may be used or actuated to manually deploy the delivery device 2506 and/or to remove the one or more anchoring elements 2502 from the delivery device 2506. The lumen of the catheter and/or elongate tube may be configured to house the delivery device 2506 wrapped at least in part with one or more pre-formed knot sutureform anchors 2502. In some examples, the catheter and/or elongate tube presents a relatively low profile. For example, a catheter may have a diameter of approximately 3 mm or less (e.g., about 9 Fr or less).

The delivery device 2506 may be used to deliver “bulky knot” type tissue anchors. For example, the delivery device 2506 may be utilized to deliver one or more of the anchoring elements 2502 to a distal side of a first valve leaflet and/or a second valve leaflet. In some instances, an elongate pusher may be movably disposed within a lumen of the catheter and/or elongate tube and/or coupled to a pusher hub that may be movably disposed within a handle and/or releasably coupled to the plunger. The delivery device 2506 carrying one or more pre-formed tissue anchoring element sutureforms 2502 can be movably disposed within a lumen of the pusher and coupled to a needle hub that is also coupled to the plunger. The plunger can be used to actuate or move the needle and the pusher during deployment of a distal anchoring element 2502 (e.g., the first anchoring element 2502a) and is movably disposed at least partially within the handle. For example, the handle may define a lumen in which the plunger can be moved. During operation, the pusher may also move within the lumen of the handle. A plunger lock can be used to prevent the plunger from moving within the handle during storage and prior to performing a procedure to deploy a tissue anchor.

Advancement of the delivery device 2506 may be performed in conjunction with echo imaging, direct visualization (e.g., direct transblood visualization), and/or any other suitable remote visualization technique/modality. With respect to cardiac procedures, for example, the delivery device 2506 may be advanced in conjunction with transesophageal (TEE) guidance and/or intracardiac echocardiography (ICE) guidance to facilitate and to direct the movement and proper positioning of the device for contacting the appropriate target cardiac region and/or target cardiac tissue (e.g., a valve leaflet, a valve annulus, or any other suitable cardiac tissue). In some instances, the delivery device 2506 may be configured to penetrate (e.g., with a pointed tip of the delivery device 2506) a first leaflet of a heart valve (e.g., the mitral valve) within a ventricle (e.g., the left ventricle) and/or a second leaflet of the heart valve.

FIG. 26 illustrates a pusher 2620 which may be configured to deploy one or more anchoring elements at one or more valve leaflets in accordance with one or more instances. A pusher 2620 may include any means for pushing and/or means for receiving one or more suture anchors. In some instances, the pusher 2620 may comprise a main body 2623 portion and/or an elastic tip 2621 portion. The main body 2623 may be sized and/or otherwise configured to receive and/or fit over a delivery device (e.g., a needle) and/or a sutureform wrapped around the delivery device. In some instances, the main body 2623 portion may be configured to receive two or more sutureforms. For example, the main body 2623 and/or the elastic tip 2621 may be configured to surround a first suture anchoring element and a second anchoring element. The pusher 2620 may be configured to be retracted to expose the first anchoring element and/or to allow the first anchoring element to exit through a distal end portion 2629 of the elastic tip 2621. The elastic tip 2621 may be configured to press against the first suture anchoring element while the second suture anchoring element remains within the main body 2623 and/or within the elastic tip 2621.

The elastic tip 2621 may be configured to expand to receive sutureforms and/or to allow sutureforms situated within the main body 2623 to exit through a distal end portion 2629 of the elastic tip 2621. In some instances, the elastic tip 2621 may be configured to assume a smaller diameter (e.g., at the distal end portion 2629) than the main body 2623 when the elastic tip 2621 is not pressed outwardly (e.g., by a sutureform situated within a lumen of the elastic tip 2621 and/or pusher 2620). Accordingly, when the elastic tip 2621 is pressed against a sutureform, the elastic tip 2621 may be configured to exert a pushing force on the sutureform. The natural and/or pre-shaped diameter of the elastic tip 2621 (e.g., at the distal end portion 2629) may be slightly larger than a diameter of a delivery device (e.g., needle) configured to carry the pusher 2620. However, the diameter at the distal end portion 2629 may be very close to the diameter of the delivery device such that the diameter at the distal end portion 2629 of the elastic tip 2621 may be smaller than a combined diameter of a sutureform wrapped around the delivery device (e.g., a diameter of the delivery devices plus 2× the width of the suture forming the sutureform). The end portion 2629 may have a generally flat shape to enable the pusher 2620 to push a suture anchoring element off of the delivery device.

The elastic tip 2621 may comprise one or more expansion slits 2625 which may be configured to allow the elastic tip 2621 to expand from a natural form in which the diameter of the elastic tip 2621 may be smaller than a diameter of the main body 2623. For example, the elastic tip 2621 may comprise four expansion slits 2625. One or more of the expansion slits 2625 may represent cuts and/or separations in the elastic tip 2621 extending from the distal end portion 2629 to the main body 2623. The expansion slits 2625 may form disconnected sections of the elastic tip 2621 to allow the disconnected sections to be pressed outwardly from within a lumen of the elastic tip 2621 to allow an increase in diameter of the elastic tip 2621.

In some instances, the elastic tip 2621 may naturally form a tapered shape which may gradually increase in diameter from the distal end portion 2629 to the main body 2623. The main body 2623 may comprise a maximum diameter of the pusher 2620 and/or the distal end portion 2629 of the elastic tip 2621 may comprise a minimum diameter of the pusher 2620. The elastic tip 2621 may be configured to expand such that a diameter at the end portion 2629 may be approximately equivalent to a diameter of the main body 2623.

The pusher 2620 may be composed of an expandable core 2622 which may be surrounded and/or encapsulated by an elastic sleeve 2624. The elastic sleeve 2624 may be configured to facilitate a spring force and/or elasticity of the expandable core 2622 due at least in part to an elasticity of the elastic sleeve 2624. For example, the elastic sleeve 2624 and/or expandable core 2622 may be shape-set in the shape shown in FIG. 26. The one or more expansion slits 2625 may allow the elastic tip 2621 to expand, however the elasticity of the expandable core 2622 and/or elastic sleeve 2624 may cause the pusher 2620 to naturally return and/or attempt to return to the shape shown in FIG. 26 when expansion force is removed. In some instances, the pusher 2620 may not include an elastic sleeve 2624 and/or the expandable core 2622 may be configured to independently return to the shape shown in FIG. 26.

In some instances, the pusher 2620 may be at least partially composed of various materials. For example, the expandable core 2622 may be at least partially composed of polycarbonate and/or the elastic sleeve 2624 may be at least partially composed of silicone.

### Valve Repair Processes

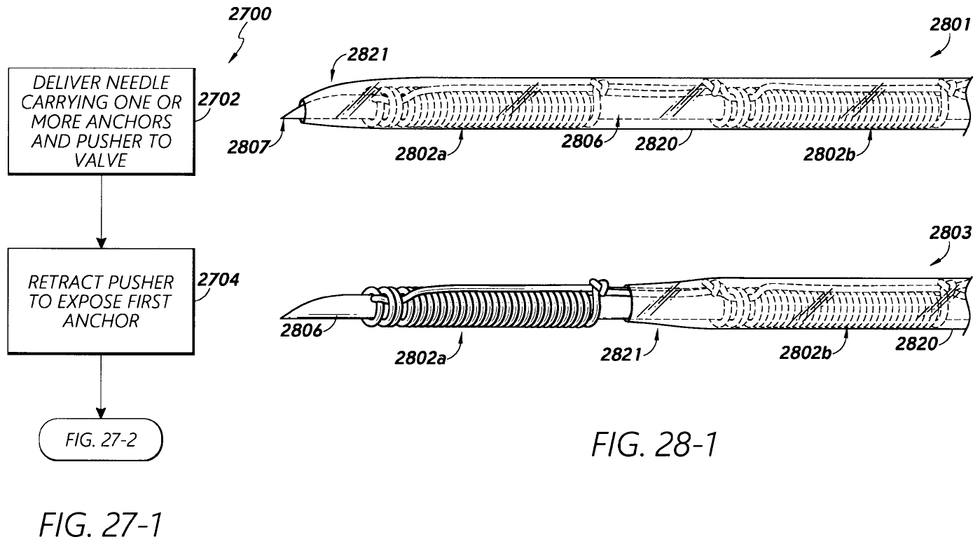

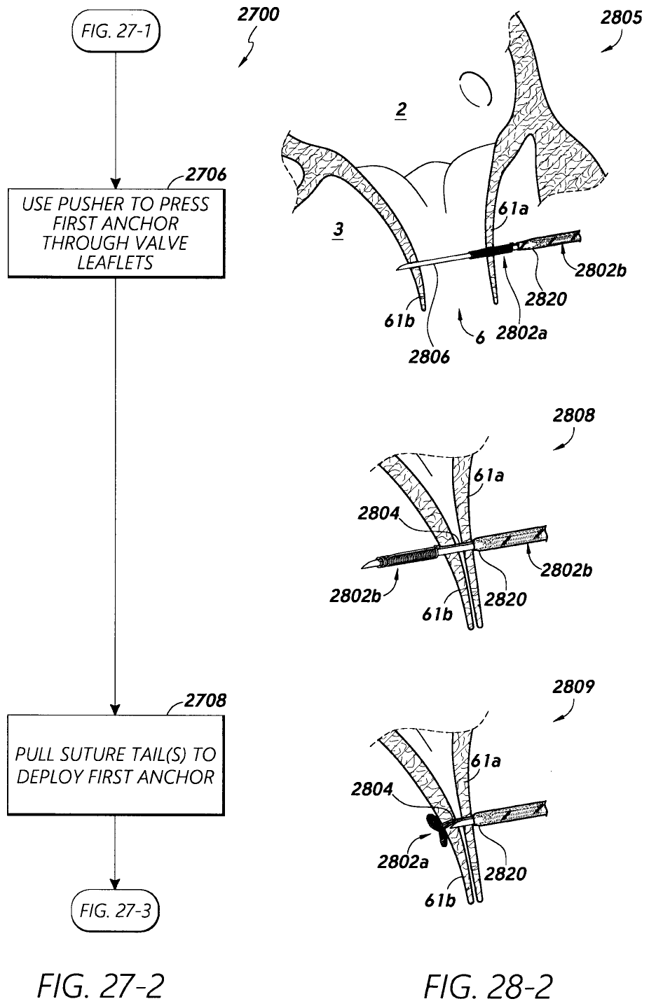

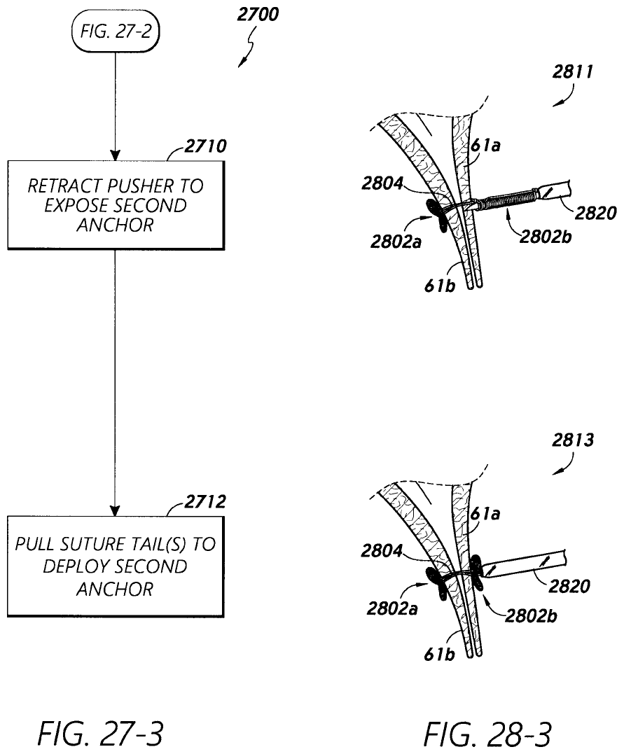

FIG. 27 (27-1, 27-2, and 27-3) provides a flow diagram representing a process 2700 for delivery of one or more anchoring elements to a valve of a heart according to one or more instances disclosed herein. FIG. 28 (28-1, 28-2, and 28-3) shows examples of various stages of the process 2700 for delivery of one or more anchoring elements to a valve of a heart shown in FIG. 27.

At step 2702, the process 2700 involves delivering a delivery device 2806 (e.g., a needle) carrying one or more anchoring elements 2802 and/or a pusher 2820 to a valve (e.g., a mitral valve) of a heart, as shown in images 2801 and 2803 of FIG. 28. The delivery device 2806 may comprise a pointed tip 2807 configured to penetrate a valve leaflet to deliver one or more anchoring elements 2802 to a distal side of the valve leaflet. At delivery, the pusher 2820 may be configured to receive and/or cover a first anchoring element 2802a and/or a second anchoring element 2802b. For example, the first anchoring element 2802a and/or second anchoring element 2802b may be situated within a lumen of the pusher 2820. An elastic tip 2821 of the pusher 2820 may be configured to be situated at or near the tip 2807 of the delivery device 2806.

The delivery device 2806 and/or one or more coaptation devices may be inserted into the heart via a catheter and/or other percutaneous procedure. For example, the delivery device 2806 may be delivered using a transfemoral, transendocardial, transcoronary, transseptal, and/or transapical procedure, or other approach. In optional instances, the delivery device and/or one or more coaptation devices may be introduced into the desired location during an open-chest surgical procedure, or using other surgical or non-surgical techniques known in the art.

At step 2704, the process 2700 involves retracting (e.g., pulling back) the pusher 2820 to expose the first anchoring element 2802a and/or to remove the first anchoring element 2802a from a lumen of the pusher 2820, as shown in image 2803 of FIG. 28. The elastic tip 2821 of the pusher 2820 may be configured to expand to allow the first anchoring element 2802a to press the elastic tip 2821 outwardly and/or allow the first anchoring element 2802a to slide through the elastic tip 2821 of the pusher 2820. The elastic tip 2821 may be configured to naturally at least partially assume a diameter that is less than a diameter of the first anchoring element 2802a while the first anchoring element 2802a is situated on the delivery device 2806. Thus, the elastic tip 2821 may at least partially expand during retraction of the pusher 2820 and/or removal of the first anchoring element 2802a from the lumen of the pusher 2820. When the first anchoring element 2802a is completely and/or at least partially removed from the pusher 2820, the elastic tip 2821 may be configured to at least partially compress such that at least a portion of the elastic tip 2821 may have a diameter that is less than the diameter of the first anchoring element 2802a while the first anchoring element 2802a is situated on the delivery device 2806. The second anchoring element 2802b may remain at least partially within the pusher 2820 following exposure and/or removal of the first anchoring element 2802a.

At step 2706, the process 2700 involves using the pusher 2820 to press against the first anchoring element 2802a and/or to press the first anchoring element 2802a towards the tip 2807 of the delivery device 2806 and/or through one or more leaflets 61 (e.g., a first leaflet 61a) of the valve, as shown in image 2805 of FIG. 28. The reduced diameter of the elastic tip 2821 of the pusher 2820 may be configured to prevent the pusher 2820 from passing over the first anchoring element 2802a when the pusher 2820 is pressed against the first anchoring element 2802a. Accordingly, the pusher 2820 may be configured to apply a pushing force to the first anchoring element 2802a to move the first anchoring element 2802a along and/or off of the delivery device 2806. The tip 2807 of the delivery device 2806 may be passed through a first leaflet 61a and/or a second leaflet 61b of the valve 6.

The pusher 2820 may press against the first anchoring element 2802a and/or the delivery device 2806 may be extended until the first anchoring element 2802a, but not the pusher 2820 and/or second anchoring element 2802b, is situated at a distal side of the first leaflet 61a and/or the second leaflet 61b, as shown in image 2808 of FIG. 28. In some instances, the pusher 2820 may be pressed against the first anchoring element 2802a while simultaneously and/or sequentially the delivery device 2806 is extended to carry the first anchoring element 2802a to the distal side of the first leaflet 61a and/or the second leaflet 61b. The diameter of the pusher 2820 may prevent the pusher 2820 and/or second anchoring element 2802b from passing through an opening in the first leaflet 61a created by the delivery device 2806. In some instances, the pusher 2820 may be configured to press against the first leaflet 61a to cause the first leaflet 61a to move towards and/or press against the second leaflet 61b. The resistance created by the first leaflet 61a and second leaflet 61b being pressed together may be sufficient to prevent the first anchoring element 2802a from retracting back through the first leaflet 61a and/or second leaflet 61b.

At step 2708, the process 2700 involves deploying the first anchoring element 2802a such that the first anchoring element 2802a may form a bulky knot form, as shown in image 2809 of FIG. 28. In some instances, deploying the first anchoring element 2802a may involve pulling one or more suture tails 2804 extending from the first anchoring element 2802 and/or retracting the delivery device 2806 to remove the first anchoring element 2802a from the delivery device 2806. The suture tails 2804 may be configured to form both the first anchoring element 2802a and the second anchoring element 2802b. The suture tails 2804 may represent tethering sutures configured to tether the first anchoring element 2802a to the second anchoring element 2802b.

At step 2710, the process 2700 involves retracting (e.g., pulling back) the pusher 2820 to expose the second anchoring element 2802b and/or to remove the second anchoring element 2802b from a lumen of the pusher 2820, as shown in image 2811 of FIG. 28. The elastic tip 2821 of the pusher 2820 may be configured to expand to allow the second anchoring element 2802b to press the elastic tip 2821 outwardly and/or allow the second anchoring element 2802b to slide through the elastic tip 2821 of the pusher 2820. The elastic tip 2821 may be configured to naturally at least partially assume a diameter that is less than a diameter of the second anchoring element 2802b while the second anchoring element 2802b is situated on the delivery device 2806. Thus, the elastic tip 2821 may at least partially expand during retraction of the pusher 2820 and/or removal of the second anchoring element 2802b from the lumen of the pusher 2820. When the second anchoring element 2802b is completely and/or at least partially removed from the pusher 2820, the elastic tip 2821 may be configured to at least partially compress due to elasticity of the elastic tip 2821 such that at least a portion of the elastic tip 2821 may have a diameter that is less than the diameter of the second anchoring element 2802b while the second anchoring element 2802b is situated on the delivery device 2806. The second anchoring element 2802b may be situated at a proximal side of the first leaflet 61a when the second anchoring element 2802b is removed from the pusher 2820.

At step 2712, the process 2700 involves deploying the second anchoring element 2802b such that the second anchoring element 2802b may form a bulky knot form, as shown in image 2813 of FIG. 28. In some instances, deploying the second anchoring element 2802b may involve pulling one or more suture tails 2804 extending from the second anchoring element 2802b and/or retracting the delivery device 2806 to remove the second anchoring element 2802b from the delivery device 2806. The suture tails 2804 may be configured to form both the first anchoring element 2802a and the second anchoring element 2802b.

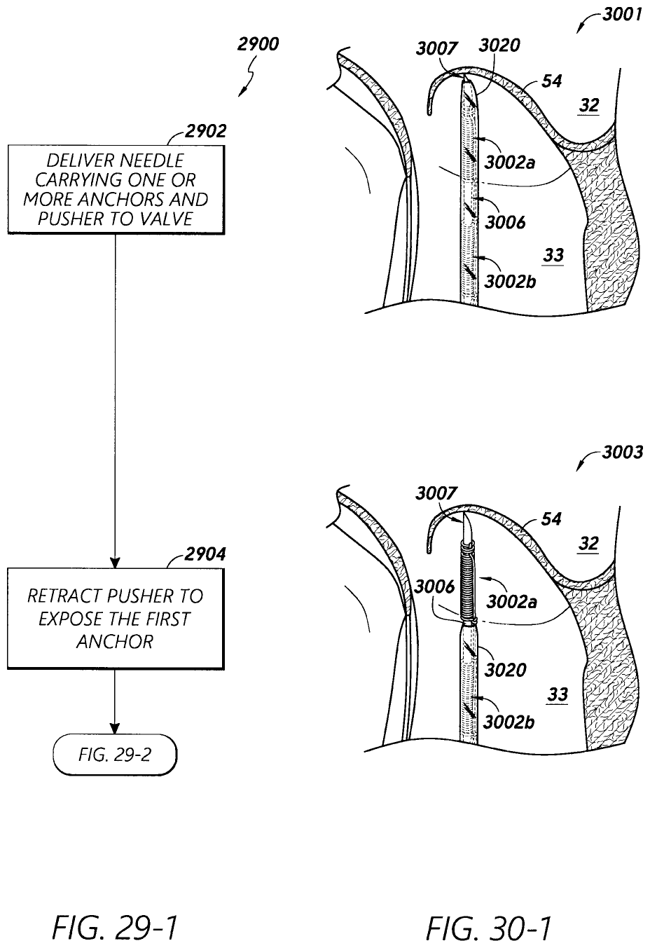

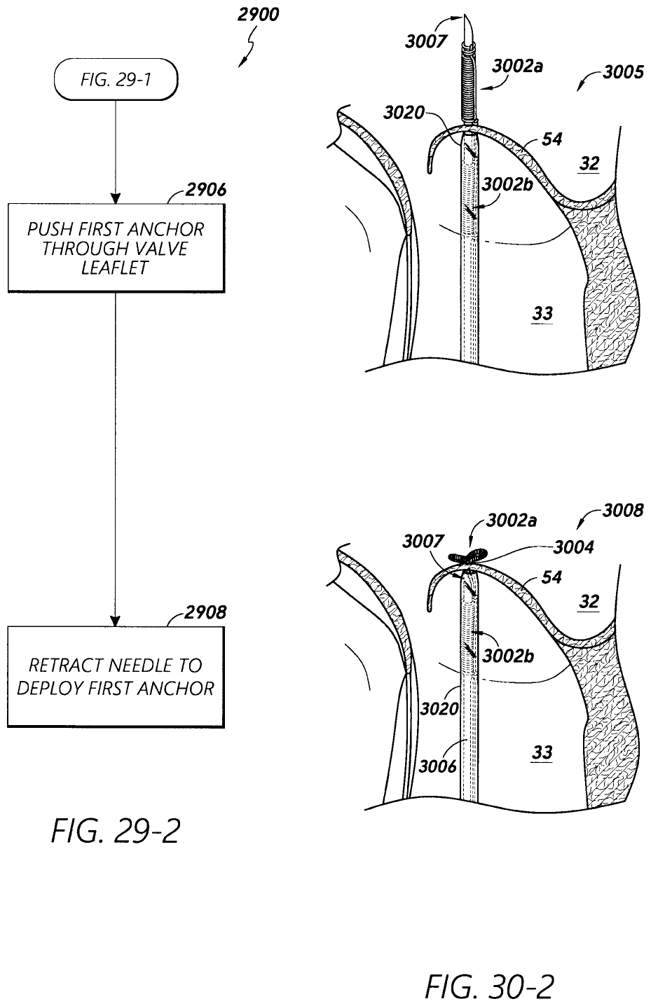

FIG. 29 (29-1 and 29-2) provides a flow diagram representing a process 2900 for delivery of one or more anchoring elements to a valve of a heart according to one or more instances disclosed herein. FIG. 30 (30-1 and 30-2) shows examples of various stages of the process 2900 for delivery of one or more anchoring elements to a valve of a heart shown in FIG. 29. While steps of FIG. 30 are shown for simplicity without an end effector and/or shaft for delivering the delivery device 3006, such devices may be used in delivery of the various anchoring elements (see, e.g., FIG. 5).

At step 2902, the process 2900 involves delivering a delivery device 3006 (e.g., a needle) carrying one or more anchoring elements 3002 and/or a pusher 3020 to a valve and/or valve leaflet 54 of a heart, as shown in image 3001 of FIG. 30. The delivery device 3006 may comprise a pointed tip 3007 configured to penetrate a valve leaflet to deliver one or more anchoring elements 3002 to a distal side of the valve leaflet. At delivery, the pusher 3020 may be configured to cover a first anchoring element 3002a and/or a second anchoring element 3002b. For example, the first anchoring element 3002a and/or second anchoring element 3002b may be situated within a lumen of the pusher 3020. An elastic tip of the pusher 3020 may be configured to be situated at or near the tip 3007 of the delivery device 3006. The delivery device 3006 may be delivered via a ventricle 33 (e.g., the left ventricle) to an underside of the leaflet 54.

The delivery device 3006 and/or one or more coaptation devices may be inserted into the heart via a catheter and/or other percutaneous procedure. For example, the delivery device 3006 may be delivered using a transfemoral, transendocardial, transcoronary, transseptal, and/or transapical procedure, or other approach. In optional instances, the delivery device and/or one or more coaptation devices may be introduced into the desired location during an open-chest surgical procedure, or using other surgical or non-surgical techniques known in the art.

At step 2904, the process 2900 involves retracting (e.g., pulling back) the pusher 3020 to expose the first anchoring element 3002a and/or to remove the first anchoring element 3002a from a lumen of the pusher 3020, as shown in image 3003 of FIG. 30. The elastic tip of the pusher 3020 may be configured to expand to allow the first anchoring element 3002a to press the elastic tip outwardly and/or allow the first anchoring element 3002a to slide through the elastic tip of the pusher 3020. The elastic tip may be configured to naturally at least partially assume a diameter that is less than a diameter of the first anchoring element 3002a while the first anchoring element 3002a is situated on the delivery device 3006. Thus, the elastic tip may at least partially expand during retraction of the pusher 3020 and/or removal of the first anchoring element 3002a from the lumen of the pusher 3020. When the first anchoring element 3002a is completely and/or at least partially removed from the pusher 3020, the elastic tip may be configured to at least partially compress such that at least a portion of the elastic tip may have a diameter that is less than the diameter of the first anchoring element 3002a while the first anchoring element 3002a is situated on the delivery device 3006. The second anchoring element 3002b may remain at least partially within the pusher 3020 following exposure and/or removal of the first anchoring element 3002a.

At step 2906, the process 2900 involves extending the delivery device 3006 such that at least the tip 3007 of the delivery device 3006 passes through the leaflet 54 and/or pressing the pusher 3020 against the first anchoring element 3002a such that the first anchoring element 3002a passes at least partially through the opening in the leaflet 54 created by the delivery device 3006, as shown in image 3005 of FIG. 30. The reduced diameter of the elastic tip of the pusher 3020 may be configured to prevent the pusher 3020 from passing over the first anchoring element 3002a when the pusher 3020 is pressed against the first anchoring element 3002a. Accordingly, the pusher 3020 may be configured to apply a pushing force to the first anchoring element 3002a to move the first anchoring element 3002a along and/or off of the delivery device 3006. The tip 3007 of the delivery device 3006 may be passed through the leaflet 54 such that the tip 3007 and/or the first anchoring element 3002a may be at least partially situated within an atrium 32 (e.g., the left atrium).

The pusher 3020 may press against the first anchoring element 3002a and/or the delivery device 3006 may be extended until the first anchoring element 3002a, but not the pusher 3020 and/or second anchoring element 3002b, is situated at an upper and/or distal side of the leaflet 54, as shown in image 3005 of FIG. 30. In some instances, the pusher 3020 may be pressed against the first anchoring element 3002a while simultaneously and/or sequentially the delivery device 3006 is extended to carry the first anchoring element 3002a to the upper/distal side of the leaflet 54. The diameter of the pusher 3020 may prevent the pusher 3020 and/or second anchoring element 3002b from passing through an opening in the first leaflet 61a created by the delivery device 3006.

At step 2908, the process 2900 involves deploying the second anchoring element 3002b such that the second anchoring element 3002b may form a bulky knot form, as shown in image 3008 of FIG. 30. In some instances, deploying the second anchoring element 3002b may involve pulling one or more suture tails 3004 extending from the second anchoring element 3002b and/or retracting the delivery device 3006 to remove the second anchoring element 3002b from the delivery device 3006.

The steps of FIG. 29 may be repeated for delivering additional anchoring elements 3002, including the second anchoring element 3002b, through the leaflet 54 and/or a different leaflet. In some instances, the delivery device 3006 may be configured to simultaneously carry two or more anchoring elements 3002. For example, the delivery device 3006 may be configured to carry six or more anchoring elements 3002. In some instances, each of the anchoring elements 3002 may be composed of different sutures and/or lengths of suture such that one or more anchoring elements 3002 may be deployed independently of other anchoring elements 3002. By carrying multiple anchoring elements 3002, the delivery device 3006 may advantageously not be required to be removed from the body following delivery of the first anchoring element 3002a and/or second anchoring element 3002b. For example, the delivery device 3006 may be configured to remain within the ventricle 33 until all anchoring elements 3002 (e.g., six anchoring elements 3002) have been delivered to an upper/distal side of the leaflet 54 and/or a different leaflet.

After deploying the first anchoring element 3002a, the second anchoring element 3002b may be moved towards the tip 3007 of the delivery device 3006 to prepare the second anchoring element 3002b to be deployed. This process may be repeated for each anchoring element 3002 carried by the delivery device 3006. In some instances, a second pusher (not shown) may be used to press against a last anchoring element 3002 carried by the delivery device 3006. For example, the second pusher may be configured to press against a sixth anchoring element 3002 to cause the sixth, fifth, fourth, third, and/or second anchoring elements 3002 to move towards the tip 3007 of the delivery device 3006 after the first anchoring element 3002a is deployed.

The suture tails 3004 extending from the one or more anchoring elements 3002 may be configured to form artificial chordae for the valve leaflet 54. For example, one or more suture tails 3004 may be anchored to a ventricle wall of the ventricle 33 to create tension at the anchoring elements 3002 at the upper/distal side of the leaflet 54.

The process shown in FIGS. 29 and 30 and/or other processes, devices, and systems disclosed herein may advantageously provide mechanisms for implementing valvular repairing using a fully transcatheter procedure on a beating heart. Furthermore, in certain instances, the various devices may be designed to be retrievable.

FIG. 31 illustrates an overhead view of an upper/distal side of a valve 3111 (e.g., viewing the mitral valve from the left atrium) having two anchoring elements 3102 with a bulky knot form delivered to the upper/distal side of the valve 3111. As shown in FIG. 31, a first anchoring element 3102a may be delivered to a first leaflet 54 and/or a second anchoring element 3102b may be delivered to a second leaflet 52. However, any number of anchoring elements 3102 may be delivered to either leaflet. For example, the first anchoring element 3102a and second anchoring element 3102b may alternatively both be delivered to the first leaflet 54.

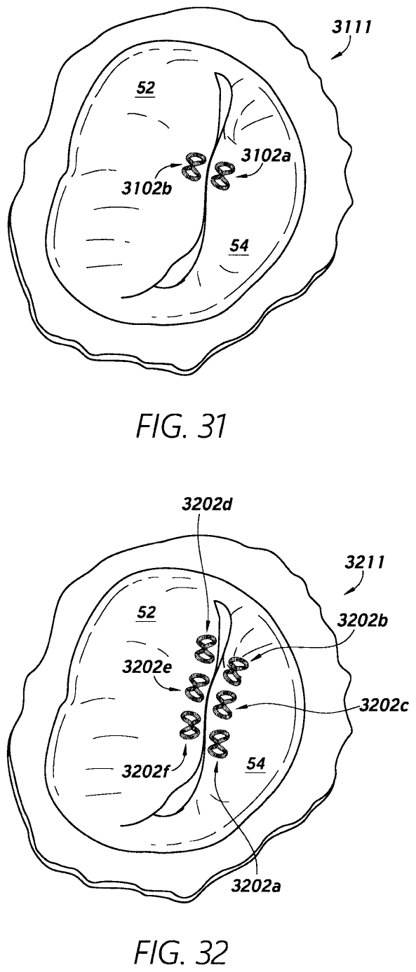

FIG. 32 illustrates an overhead view of an upper/distal side of a valve 3211 (e.g., viewing the mitral valve from the left atrium) having six anchoring elements 3202 with a bulky knot form delivered to the upper/distal side of the valve 3211. In some instances, the six anchoring elements 3202 may be delivered simultaneously via a single delivery device (e.g., a needle) and/or may be deployed sequentially through and/or at the valve 3211. As shown in FIG. 32, a first anchoring element 3202a, second anchoring element 3202b, and/or third anchoring element 3202c may be delivered to a first leaflet 54 and/or a fourth anchoring element 3202d, fifth anchoring element 3202e, and/or sixth anchoring element 3202f may be delivered to a second leaflet 52. However, any number of anchoring elements 3202 may be delivered to either leaflet.

### Additional Instances

Depending on the instance, certain acts, events, or functions of any of the processes or algorithms described herein can be performed in a different sequence, may be added, merged, or left out altogether. Thus, in certain instances, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain instances include, while other instances do not include, certain features, elements and/or steps. Thus, such conditional language is not generally intended to imply that features, elements and/or steps are in any way required for one or more instances or that one or more instances necessarily include logic for deciding, with or without author input or prompting, whether these features, elements and/or steps are included or are to be performed in any particular instance. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain instances require at least one of X, at least one of Y and at least one of Z to each be present.

It should be appreciated that in the above description of instances, various features are sometimes grouped together in a single instance, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular instance herein can be applied to or used with any other instance(s). Further, no component, feature, step, or group of components, features, or steps are necessary or indispensable for each instance. Thus, it is intended that the scope of the inventions herein disclosed and claimed below should not be limited by the particular instances described above, but should be determined only by a fair reading of the claims that follow.

What is claimed is:

1. A method comprising:
inserting a needle into a ventricle of a heart, the needle configured to deliver a first anchoring element, a second anchoring element, and a tethering suture to the ventricle, the tethering suture tethered between the first anchoring element and the second anchoring element;
penetrating a first leaflet of a heart valve with the needle;
penetrating a second leaflet of the heart valve with the needle;
deploying the first anchoring element at a distal side of the second leaflet;
retracting the needle from the first leaflet and the second leaflet;
deploying the second anchoring element at a proximal side of the first leaflet;
cinching the tethering suture to cause a desired amount of valve coaptation; and
locking the tethering suture.

2. The method of claim 1, wherein the needle is inserted into the ventricle via a septum.

3. The method of claim 1, wherein the needle is inserted into the ventricle via a posterior wall of the heart.

4. The method of claim 1, wherein the first anchoring element comprises a first sutureform wrapped at least partially around the needle.

5. The method of claim 4, wherein the tethering suture extends from the first sutureform.

6. The method of claim 4, wherein the second anchoring element comprises a second sutureform.

7. The method of claim 6, wherein the tethering suture is configured to pass at least partially through a lumen formed by coils of the second sutureform.

8. The method of claim 1, further comprising extending a pusher along an outer surface of the needle to press the first anchoring element and the second anchoring element off of the needle.

9. The method of claim 1, wherein the needle comprises an internal lumen and an aperture to the internal lumen.

10. The method of claim 1, wherein the tethering suture is further configured to form a first knot between the first anchoring element and the second anchoring element.

11. The method of claim 2, wherein the tethering suture is further configured to form a second knot between the first anchoring element and the second anchoring element.

12. The method of claim 1, wherein the first anchoring element is configured to form a non-continuous circular shape when removed from the needle.

13. The method of claim 12, wherein the first anchoring element comprises:
a base portion;
a shaping device configured to fit within the base portion; and
two or more appendages extending from the base portion;
wherein the tethering suture is configured to pass through each of the two or more appendages to pull the two or more appendages radially inward towards a central area of the first anchoring element.

14. The method of claim 1, wherein the first anchoring element, the second anchoring element, and the tethering suture are formed from a single suture.

15. A system comprising:
a first anchoring element;
a second anchoring element;
a tethering suture tethered between the first anchoring element and the second anchoring element; and
a needle configured to:
penetrate a first leaflet of a heart valve within a ventricle of a heart;
penetrate a second leaflet of the heart valve;
deliver the first anchoring element at a distal side of the second leaflet; and
deliver the second anchoring element at a proximal side of the first leaflet.

16. The system of claim 15, wherein the first anchoring element comprises a first sutureform wrapped at least partially around the needle.

17. The system of claim 15, further comprising a pusher configured to extend along an outer surface of the needle to press the first anchoring element and the second anchoring element off of the needle.

18. The system of claim 15, wherein:
the needle comprises an internal lumen and an aperture to the internal lumen; and
the needle is further configured to deliver the first anchoring element via the internal lumen and the aperture.

19. The system of claim 15, wherein the first anchoring element is configured to form a non-continuous circular shape when removed from the needle.

20. The system of claim 19, wherein the first anchoring element comprises:
a base portion;
a shaping device configured to fit within the base portion; and
two or more appendages extending from the base portion;
wherein the tethering suture is configured to pass through each of the two or more appendages to pull the two or more appendages radially inward towards a central area of the first anchoring element.
